(19) United States
(12) Patent Application Publication
(10) Pub. No.: US 2015/0065724 A1
(43) Pub. Date: Mar. 5, 2015

(54) DIASTEREOSELECTIVE METHODS FOR SYNTHESIZING COMPOUNDS

(71) Applicant: AbbVie Inc., North Chicago, IL (US)

(72) Inventors: Yi-Yin Ku, Buffalo Grove, IL (US); Yu-Ming Pu, Gurnee, IL (US); Hao Yang, Lake Bluff, IL (US); Alan Christesen, Round Lake Beach, IL (US)

(73) Assignee: AbbVie Inc., North Chicago, IL (US)

(21) Appl. No.: 14/475,748

(22) Filed: Sep. 3, 2014

Related U.S. Application Data
(60) Provisional application No. 61/873,939, filed on Sep. 5, 2013.

(51) Int. Cl. C07D 413/04; C07F 7/08

(52) U.S. Cl. CPC C07D 413/04; C07F 7/0812
USPC 548/110; 548/240

(57) ABSTRACT

The present invention is directed to novel synthetic methods for preparing a compound of Structural Formula (I):

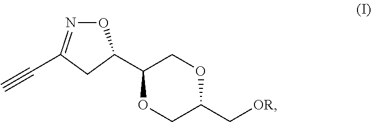

wherein R is —H or a hydroxyl protecting group. Also included are synthetic intermediates described herein.

### REFERENCE TO RELATED APPLICATION

This application claims priority to and the benefit of the filing date under 35 U.S.C. 119(e) to U.S. Provisional Application No. 61/873,939, filed on Sep. 5, 2013, the entire content of which is incorporated herein by reference.

### BACKGROUND OF THE INVENTION

Matrix metalloproteinases (MMPs) are a family of structurally related zinc-containing enzymes that have been reported to mediate the breakdown of connective tissue in normal physiological processes such as embryonic development, reproduction and tissue remodeling as well as pathological conditions such as rheumatoid arthritis (RA), osteoarthritis (OA), osteoporosis, atherosclerosis and tumor metastasis. MMP family comprises of more than 20 members in human including collagenases (MMP-1, MMP-8, MMP-13), gelatinases (MMP-2, MMP-9), stromelysins (MMP-3, MMP-10, MMP-11), matrilysins (MMP-7, MMP-26), membrane-type (MMP-14, MMP-15, MMP-16, MMP-17, MMP-24, MMP-25), as well as metalloelastases (MMP-12, MMP-19, MMP-20, MMP-22, MMP-23) (Nat. Rev. Drug Discov., 2007, 6:480-498).

The most significant members of the MMP family with respect to OA pathology are the collagenases (MMP-1, -8, and -13) which are responsible for type II collagen breakdown (Nat. Rev. Drug Discov., 2007, 6:480-498; Semin. Cell Dev. Biol., 2008, 19:61-68). In recent years, increasing evidence suggests that MMP-13 is the main collagenase responsible for degradation of type II collagen in OA. MMP-13 is not found in normal adult tissues but is specifically expressed in the articular cartilage of OA patients (J. Rheumatol., 1996, 23:590-595; J. Clin. Invest. 1996, 97:2011-2019; J. Clin. Invest., 1996, 97:761-768; J. Clin. Invest., 1997, 99:1534-45). Analysis of human OA cartilage shows a correlation between presence of MMP-13 and MMP-specific collagen cleavage products with disease severity (Arthritis Rheum., 1983, 26:63-8; J. Rheumatol., 2005, 32:876-886). In vitro data demonstrate that MMP-13 selective inhibitors prevent cytokine-induced collagen loss in human and bovine cartilage ex-plant cultures (Arthritis Rheum. 2009, 60:2008-2018; J. Biol. Chem., 2007, 282:27781-27791).

Preclinical models of OA have elevated MMP-13 expression and MMP-13-induced collagen cleavage products in cartilage, synovial fluid, and urine which have been shown to correlate with disease progression (Osteoarthritis Cartilage, 2005, 13:139-145; Arthritis Rheum., 1998 41:877-890). Transgenic mice expressing active human MMP-13 through a cartilage-specific promoter demonstrate pathological changes in articular cartilage of the mouse joints similar to those observed in human OA (J. Clin. Invest., 2001, 107:35-44; Arthritis Rheum., 2003, 48:1077). In contrast, MMP-13 deficient mice show significantly reduced cartilage degradation as compared to the wild-type following destabilization of the medial meniscus (Arthritis Rheum., 2009, 60:3723-3733). Lastly, an orally active MMP-13 selective inhibitor was chondroprotective in rat medial meniscus tear (MMT), rabbit and dog anterior cruciate ligament/medial meniscectomy models of OA (Arthritis Rheum., 2009, 60:2008-2018; J. Biol. Chem., 2007, 282:27781-27791; Arthritis Rheum., 2010, 62:3006-3015). Taken together, these data indicate that MMP-13 plays an important role in development and progression of the OA in preclinical models and that selective inhibition of MMP-13 can halt breakdown of cartilage thereby preventing joint destruction.

The catalytic zinc domain in MMPs has been the primary focus of inhibitor design. The modification of substrates by introducing zinc chelating groups has generated potent inhibitors such as peptide hydroxamates and thiol-containing peptides (Drug Discov. Today, 2007, 12:640-646). Over the last 10-15 years, many non-selective MMP inhibitors have advanced to Phase II clinical trials in treatment of diseases such as cancer, rheumatoid arthritis and OA. However, none of these inhibitors have advanced to late stage trials due to a number of significant challenges: A) Highly variable pharmacokinetics and often poor oral bioavailability. B) All of these non-selective inhibitors target the zinc-binding site which is common to all matrix metalloproteinases. The clinical utility of non-selective MMP inhibitors has been restricted by dose-dependent musculoskeletal effects in humans [joint stiffness, inflammation, pain in arms and shoulders termed “musculoskeletal syndrome” (MSS)] (Arthritis Res. Ther., 2007, 9:R109). No specific MMP has been implicated in MSS and it is believed that non-selective inhibition of multiple MMPs is the primary cause of this toxicity. Although no specific MMP has been implicated in MSS, there is substantial evidence that MMP-13 does not play a major role in development of MSS. Clinical data from humans with mis-sense mutation of MMP13 are characterized by defective growth and modeling of vertebrae and long bones and do not exhibit signs of MSS (J. Clin. Invest., 2005, 115:2832-2842). Preclinical data from mice deficient of MMP-13 also demonstrate growth defects but no histological signs of fibrodysplasia (MSS) (Development, 2004, 131:5883-5895). Finally, a 2-week rat model of fibrodysplasia (MSS) study has shown that animals dosed with a highly selective MMP-13 inhibitor do not develop histological signs of fibrodysplasia as compared to animals dosed with a pan-MMP inhibitor (Arthritis Rheum., 2009, 60:2008-2018); (J. Bio. Chem., 2007, 282:27781-27791).

Some new selective MMP-13 inhibitors are disclosed in International PCT Application Publication No. WO 2012/151158 (incorporated herein by reference).

There continues to be a need to find new selective MMP-13 inhibitors with an acceptable therapeutic window making them clinically attractive in the treatment of diseases. And a need exists for methods of synthesizing these compounds, as well as their intermediates.

### SUMMARY OF THE INVENTION

One aspect of the invention provides a compound of Structural Formula (I):

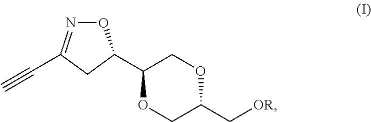

wherein R is —H or a hydroxyl protecting group.

In one embodiment, the invention provides a compound according to the previous embodiment wherein R is H, optionally substituted methyl, optionally substituted ethyl, or optionally substituted benzyl.

In one embodiment, optionally substituted methyl includes, but is not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR).

In one embodiment, optionally substituted ethyl includes, but is not limited to, ethyl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, and t-butyl.

In one embodiment, optionally substituted benzyl includes, but is not limited to, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, diphenylmethyl, p,p′-dinitrobenzhydryl, and 5-dibenzosuberyl.

In one embodiment, the invention provides a compound according to any one of the foregoing embodiments wherein R is benzyl.

In another aspect, the invention provides a method of preparing a compound of Structural Formula (I):

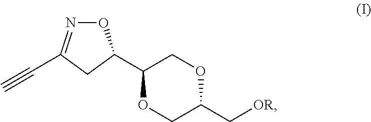

the method comprising: cyclizing a compound of Structural Formula (IIa) or (IIb):

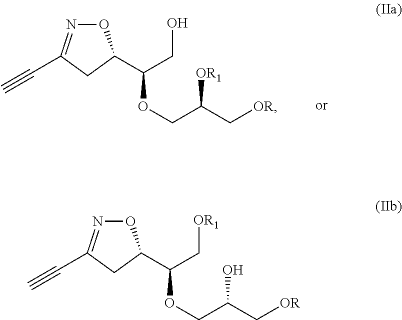

in the presence of a base, wherein R is —H, optionally substituted methyl, optionally substituted ethyl, or optionally substituted benzyl; and R₁ is a hydroxyl activation group.

The term “hydroxyl activation group” or “hydroxyl activating group” used herein refers to a group such that OR₁ will form a good leaving group during a cyclization reaction.

In one embodiment, the invention provides a method according to the previous embodiment wherein R is —H, optionally substituted methyl, optionally substituted ethyl, or optionally substituted benzyl.

In one embodiment, the invention provides a method according to any one of the previous embodiments, wherein R₁ is C₁₋₁₀ alkylsulfonate or C₁₋₁₀ arylsulfonate.

In one embodiment, the invention provides a method according to any one of the previous embodiments, R₁ is mesylate, triflate, nonaflate, tresylate, besylate, nosylate, brosylate, or tosylate.

In one embodiment, the invention provides a method according to any one of the previous embodiments wherein the base is a strong base, for example, an alkali metal hydroxide, an alkali metal C₁₋₆alkoxide (e.g., sodium tert-butoxide), or an alkali metal bis(trimethylsilyl)amide.

The term “strong base” used herein refers to a base with a pKa value greater than or equal to 14.

The term “alkali metal” as used herein refers to lithium (Li), sodium (Na), potassium (K), rubidium (Rb), and caesium (Cs).

When an alkali metal hydroxide is used as a base, a phase transfer catalyst, such as a quaternary ammonium salt (e.g., tetrabutylammonium chloride, bromide or iodide) can be used.

In one embodiment, the invention provides a method according to any one of the previous embodiments wherein R is benzyl and R₁ is tosylate.

In another aspect, the invention provides a compound of Structural Formula (III):

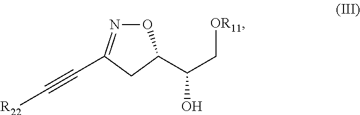

wherein R₁₁ is —H or a hydroxyl protecting group; and R₂₂ is H, trimethylsilyl (TMS), triethylsilyl (TES), triisopropylsilyl (TIPS), dimethylisopropylsilyl (DMIPS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), biphenyldimethylsilyl, triisopropylsilyl, biphenyldiisoporpylsilyl, or 2-(2-hydroxypropyl).

In one embodiment, the invention provides a compound according to the previous embodiment wherein R₁₁ is t-butyldimethylsilyl (TBDMS), t-butyldiphenylsilyl (TBDPS), or trityl.

In one embodiment, the invention provides a compound according to any one of the foregoing embodiments wherein R₂₂ is trimethylsilyl (TMS), triethylsilyl (TES), t-butyldimethylsilyl (TBDMS), dimethylthexylsilyl, biphenyldimethylsilyl, triisopropylsilyl, biphenyldiisoporpylsilyl, or 2-(2-hydroxypropyl).

In one embodiment, the invention provides a compound according to any one of the foregoing embodiments wherein R₁₁ is t-butyldiphenylsilyl (TBDPS) and R₂₂ is trimethylsilyl (TMS).

In another aspect, the invention provides a method of preparing a compound of Structural Formula (III):

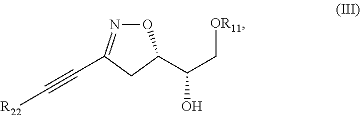

the method comprising a step of reacting a compound of Structural Formula (IV):

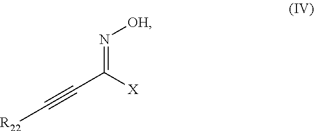

and a compound of Structural Formula (V) in the presence of a C₁₋₆alkyl magnesium halide and a C₁₋₆alcohol,

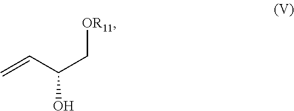

wherein:

X is halide;

R₁₁ is —H or a hydroxyl protecting group; and

R₂₂ is H, trimethylsilyl (TMS), triethylsilyl (TES), triisopropylsilyl (TIPS), dimethylisopropylsilyl (DMIPS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), biphenyldimethylsilyl, triisopropylsilyl, biphenyldiisoporpylsilyl, or 2-(2-hydroxypropyl).

In one embodiment, the invention provides a method according to the previous embodiment wherein X is chloride.

In one embodiment, the invention provides a method according to any one of the previous embodiments wherein the C₁₋₆ alkyl magnesium halide is ethylmagnesium bromide and the C₁₋₆alcohol is 2-propanol.

In one embodiment, the invention provides a method according to any one of the previous embodiments wherein R₁₁ is t-butyldimethylsilyl (TBDMS), t-butyldiphenylsilyl (TBDPS), or trityl.

In one embodiment, the invention provides a method according to any one of the previous embodiments wherein R₂₂ is trimethylsilyl (TMS), triethylsilyl (TES), t-butyldimethylsilyl (TBDMS), dimethylthexylsilyl, biphenyldimethylsilyl, triisopropylsilyl, biphenyldiisoporpylsilyl, or 2-(2-hydroxypropyl).

In one embodiment, the invention provides a method according to any one of the previous embodiments wherein R₁₁ is t-butyldiphenylsilyl (TBDPS) and R₂₂ is trimethylsilyl (TMS).

In one embodiment, the invention provides a method according to any one of the previous embodiments, further comprising a step of removing R₂₂ of the compound of Structural Formula (III), thereby forming a compound of Structural Formula (VI):

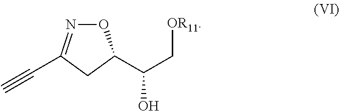

In one embodiment, the invention provides a method according to any one of the previous embodiments, wherein R₂₂ is trimethylsilyl, and is removed by water and AgNO₃.

In one embodiment, the invention provides a method according to any one of the previous embodiments, further comprising: reacting a compound of Structural Formula (VI) with a carboxylic acid R₃₃COOH to form an ester of Structural Formula (VII) via Mitsunobu inversion:

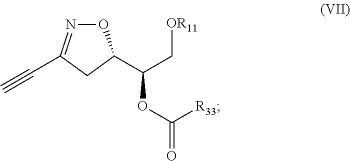

and converting the ester into an alcohol of Structural Formula (VIII):

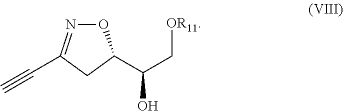

In one embodiment, the invention provides a method according to any one of the previous embodiments, wherein the reacting step is conducted in the presence of an azodicarboxylate and triphenylphosphine (TPP).

In one embodiment, the invention provides a method according to any one of the previous embodiments, wherein the azodicarboxylate is di-tert-butyl azodicarboxylate, R₃₃ is 2-pyridyl.

In one embodiment, the invention provides a method according to any one of the previous embodiments, wherein the converting step is conducted with an alcohol in the presence of Zn(OAc)₂ or Cu(OAc)₂.

In one embodiment, the invention provides a method according to any one of the previous embodiments, wherein R₃₃ is 2-pyridyl and R₁₁ is t-butyldiphenylsilyl (TBDPS).

In one embodiment, the invention provides a method according to any one of the previous embodiments, further comprising a step of reacting the compound of Structural Formula (VIII) with an epoxide

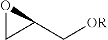

to form a compound of Structural Formula (IXa):

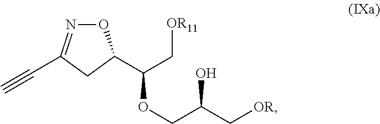

wherein R is —H or a hydroxyl protecting group.

In one embodiment, the invention provides a method according to any one of the previous embodiments, wherein the reacting step is conducted in the presence of C₁₋₆alkyl magnesium halide (e.g., ethylmagnesium bromide).

In one embodiment, the invention provides a method according to any one of the previous embodiments, wherein R is H, optionally substituted methyl, optionally substituted ethyl, or optionally substituted benzyl.

In one embodiment, the invention provides a method according to any one of the previous embodiments, R is benzyl.

In one embodiment, the invention provides a method according to any one of the previous embodiments, further comprising a step of converting the compound of Structural Formula (IXa) into a compound of Structural Formula (Xa) in the presence of an amine base:

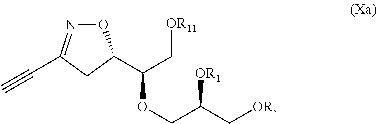

wherein R₁ is a hydroxylactivating group.

In one embodiment, the invention provides a method according to any one of the previous embodiments, wherein the amine base is N,N-diisopropylethylamine (DIPEA), triethylamine (TEA), 4-dimethylaminopyridine (DAMP), N-methylmorpholine, 1,4-diazabicyclo[2,2,2]octane (DABCO), 1,5-diazabicyclo[4,3,0]non-5-ene (DBN), or 1,8-diazabicyclo[5,4,0]undec-7-ene (DBU).

In one embodiment, the invention provides a method according to any one of the previous embodiments, wherein R₁ is C₁₋₁₀ alkylsulfonate or C₁₋₁₀ arylsulfonate.

In one embodiment, the invention provides a method according to any one of the previous embodiments, wherein R₁ is mesylate, triflate, nonaflate, tresylate, besylate, brosylate, nosylate, or tosylate.

In one embodiment, the invention provides a method according to any one of the previous embodiments, further comprising a step of converting the compound of Structural Formula (Xa) into a compound of Structural Formula (lla) in the presence of an acid:

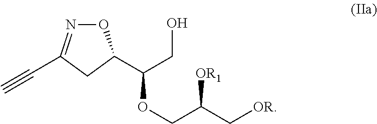

In one embodiment, the invention provides a method according to any one of the previous embodiments, wherein the acid is a carboxylic acid. In one embodiment, the carboxylic acid is formic acid, acetic acid, or propionic acid.

In one embodiment, the invention provides a method according to any one of the previous embodiments, wherein the converting step is performed in the presence of a de-silylation reagent. In one embodiment, the de-silylation reagent is a fluoride salt (e.g., tetrabutylammonium fluoride (TBAF)).

In one embodiment, the invention provides a method according to any one of the previous embodiments, wherein R₁₁ is t-butyldiphenylsilyl (TBDPS).

In one embodiment, the invention provides a method according to any one of the previous embodiments, further comprising a step of reacting the compound of Structural Formula (VIII) with an epoxide

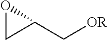

to form a compound of Structural Formula (IXb):

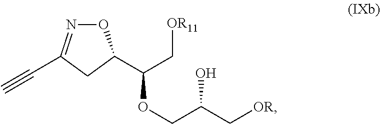

wherein R is —H or a hydroxyl protecting group.

In one embodiment, the invention provides a method according to any one of the previous embodiments, wherein the reacting step is conducted in the presence of C₁₋₆ alkyl magnesium halide (e.g. ethylmagnesium bromide).

In one embodiment, the invention provides a method according to any one of the previous embodiments, wherein R is H, optionally substituted methyl, optionally substituted ethyl, or optionally substituted benzyl. In one embodiment, R is benzyl.

In one embodiment, the invention provides a method according to any one of the previous embodiments, further comprising a step of converting the compound of Structural Formula (DO) into a compound of Structural Formula (Xb) in the presence of an acid:

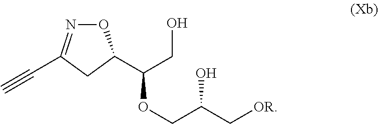

In one embodiment, the invention provides a method according to any one of the previous embodiments, wherein the acid is a carboxylic acid.

In one embodiment, the invention provides a method according to any one of the previous embodiments, the carboxylic acid is formic acid, acetic acid, or propionic acid.

In one embodiment, the invention provides a method according to any one of the previous embodiments, the converting step is performed in the presence of a fluoride salt as a de-silylation reagent. In another embodiment, the fluoride salt is tetrabutylammonium fluoride (TBAF).

In one embodiment, the invention provides a method according to any one of the previous embodiments, R₁₁ is t-butyldiphenylsilyl (TBDPS) and R is benzyl.

In one embodiment, the invention provides a method according to any one of the previous embodiments, further comprising a step of converting the compound of Structural Formula (Xb) into a compound of Structural Formula (IIb):

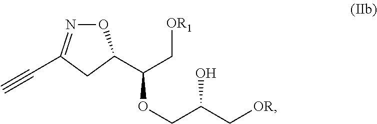

wherein R₁ is a hydroxyl activating group.

In one embodiment, the invention provides a method according to any one of the previous embodiments, wherein R₁ is C₁₋₁₀ alkylsulfonate or C₁₋₁₀ arylsulfonate.

In one embodiment, the invention provides a method according to any one of the previous embodiments, wherein R₁ is mesylate, triflate, nonaflate, tresylate, besylate, brosylate, nosylate, or tosylate.

In one embodiment, the invention provides a method according to any one of the previous embodiments, further comprising cyclizing the compound of Structural Formula (IIb) in the presence of a strong base to form a compound of Structural Formula (I):

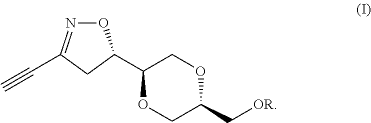

In one embodiment, the invention provides a method according to any one of the previous embodiments, wherein the strong base is an alkali metal hydroxide, an alkali metal C₁₋₆alkoxide (e.g., sodium tert butoxide), or an alkali metal bis(trimethylsilyl)amide.

In one embodiment, the invention provides a method according to any one of the previous embodiments wherein R is benzyl and R₁ is tosylate.

In one embodiment, the invention provides a method according to any one of the previous embodiments, further comprising cyclizing the compound of Structural Formula (IIa) in the presence of a strong base to form a compound of Structural Formula (I):

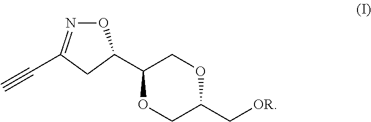

In one embodiment, the invention provides a method according to any one of the previous embodiments, wherein the strong base is an alkali metal hydroxide, an alkali metal C₁₋₆ alkoxide (e.g., sodium tert-butoxide), or an alkali metal bis(trimethylsilyl)amide.

In one embodiment, the invention provides a method according to any one of the previous embodiments wherein R is benzyl and R₁ is tosylate.

It is contemplated that any of the embodiments described herein, including embodiments only described under one aspect of the invention and embodiments only described in the examples, can be combined with one or more other embodiments where applicable.

### DETAILED DESCRIPTION OF THE INVENTION

In one aspect, the present invention relates to novel synthetic methods for preparing a compound represented by Structural Formula (I). The method comprises one or more of reaction 1, reaction 2, reaction 3, reaction 4a, reaction 4b, reaction 5a, reaction 5b, reaction 6a, reaction 6b, and/or reaction 7, as described below, or a combination thereof. For example, in one embodiment, the method comprises the steps of reaction 1. Alternatively, the method comprises the steps of reaction 1, reaction 2, and reaction 3. In another alternative, the method comprises the steps of reaction 4a or reaction 4b. In another alternative, the method comprises the steps of reaction 1, reaction 2, reaction 3, reaction 4a, reaction 5a, reaction 6a, and reaction 7. In another alternative, the method comprises the steps of reaction 1, reaction 2, reaction 3, reaction 4b, reaction 5b, reaction 6b, and reaction 7.

In one embodiment, the present invention is directed to a synthetic method (reaction 1) for preparing a compound represented by Structural Formula (III) comprising the step of reacting a compound of Structural Formula (IV) with a compound of Structural Formula (V) in the presence of a C₁₋₆alkyl magnesium halide and a C₁₋₆ alcohol:

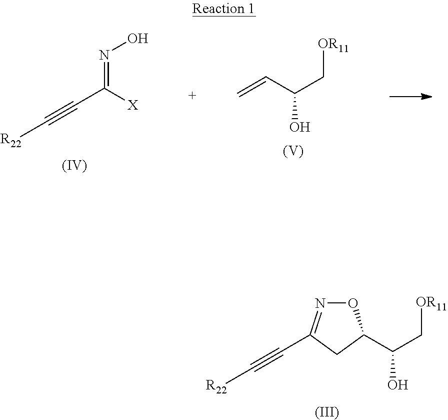

wherein X is halide; R₁₁ is —H or a hydroxyl protecting group; and R₂₂ is H, trimethylsilyl (TMS), triethylsilyl (TES), triisopropylsilyl (TIPS), dimethylisopropylsilyl (DMIPS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), biphenyldimethylsilyl, triisopropylsilyl, biphenyldiisoporpylsilyl, or 2-(2-hydroxypropyl).

In one embodiment, at least 45%, 50%, 55%, 60%, 65%, 70%, 75%, 80%, 90%, 95%, 96%, 97%, 98%, 99%, 99.5%, 99.8%, 99.9% or 100% by weight of the compound obtained by reaction 1 is represented by Structural Formula (III).

As used herein, a “hydroxyl protecting group” is a functional group that protects a hydroxyl group from participating in reactions that are occurring in other parts of the molecule. Suitable hydroxyl protecting groups are well known to those of ordinary skill in the art and include those found in T. W. Greene, Protecting Groups in Organic Synthesis, John Wiley & Sons, Inc. 3^{rd} ed.1999, the entire teachings of which are incorporated herein by reference. Exemplary hydroxyl protecting groups include, but are not limited to, optionally substituted methyl ethers (e.g., methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl); optionally substituted ethyl ethers (e.g., ethyl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl); optionally substituted benzyl ethers (e.g., benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido); silyl ethers (e.g., trimethylsilyl (TMS), triethylsilyl (TES), triisopropylsilyl (TIPS), dimethylisopropylsilyl (DMIPS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS)); esters (e.g., formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate)); carbonate (e.g., methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate); and others (e.g., 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, N,N,N′,N′-tetramethylphosphorodiamidate, 2-chlorobenzo ate, N-phenylcarbamate, borate, dimethylphosphinothioyl, 2,4-dinitrophenylsulfenate, sulfate, methanesulfonate (mesylate), benzylsulfonate, and tosylate (Ts).

In one embodiment, for reaction 1 described above, X is chloride.

In one embodiment, for reaction 1 described in any one of the foregoing embodiments, the C₁₋₆ alkyl magnesium halide is ethylmagnesium bromide and the C₁₋₆ alcohol is 2-propanol.

In one embodiment, for reaction 1 described in any one of the foregoing embodiments, R₁₁ is t-butyldimethylsilyl (TBDMS), t-butyldiphenylsilyl (TBDPS), or trityl.

In one embodiment, for reaction 1 described in any one of the foregoing embodiments, R₂₂ is trimethylsilyl (TMS), triethylsilyl (TES), t-butyldimethylsilyl (TBDMS), dimethylthexylsilyl, biphenyldimethylsilyl, triisopropylsilyl, biphenyldiisoporpylsilyl, or 2-(2-hydroxypropyl).

In one embodiment, for reaction 1 described in any one of the foregoing embodiments, R₁₁ is t-butyldiphenylsilyl (TBDPS) and R₂₂ is trimethylsilyl (TMS). Reaction 1 described in any one of the foregoing embodiments can be carried out in conditions similar to those described in Kanemasa et al., J. Am. Chem. Soc., 1994, 116:2324-2339; and Carreira et al., Org. Lett., 2005, 7(10):2011-2014. Both references are incorporated herein by reference.

Reaction 1 described in any one of the foregoing embodiments can be carried out in any suitable solvent or solvents. In one embodiment, the reaction is carried out in an organic solvent or solvents, such as dichloromethane (DCM), acetonitrile, or toluene.

Compound (V) can be obtained from selective protection of the primary alcohol of commercially available (R)-but-3-ene-1,2-diol, using known procedures in the art.

Compound (IV) can be prepared by reacting

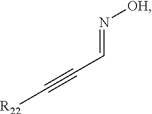

which is commercially available or, alternatively, can be synthesized as described in example 1, Step 1. with N-chlorosuccinimide (NCS) or N-bromorosuccinimide (NBS), using known procedures in the art.

In one embodiment, the present invention is also directed to a method (reaction 2) of removing R₂₂ of a compound of Structural Formula (III), thereby forming a compound of Structural Formula (VI):

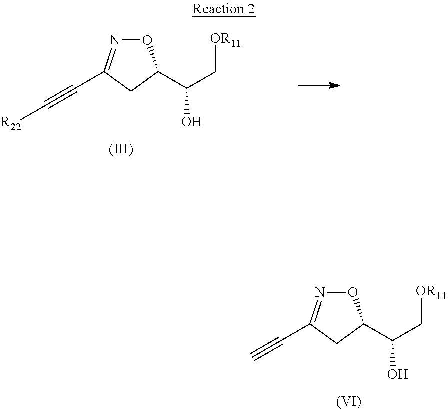

wherein values and particular values for the variables are as described above for reaction 1.

In one embodiment, at least 45%, 50%, 55%, 60%, 65%, 70%, 75%, 80%, 85%, 90%, 95%, 96%, 97%, 98%, 99%, 99.5%, 99.8%, 99.9% or 100% by weight of the compound obtained by reaction 2 is represented by Structural Formula (VI).

In one embodiment, for reaction 2 as described above, the reaction is conducted in the presence of water and AgNO₃.

In one embodiment, for reaction 2 described in any one of the foregoing embodiments is carried out in a mixture of water and an organic solvent. Suitable organic solvent includes, for example, methanol, ethanol, 1-propanol, 2-propanol, 1-butanol, tBuOH, acetone, acetonitrile or toluene.

In one embodiment, for reaction 2 described in any one of the foregoing embodiments, R₂₂ is trimethylsilyl, and removed by water and AgNO₃.

In one embodiment, the present invention is also directed to a method (reaction 3) of reacting a compound of Structural Formula (VI) with a carboxylic acid R₃₃COOH to form an ester of Structural Formula (VII) via Mitsunobu inversion; and converting the ester into an alcohol of Structural Formula (VIII).

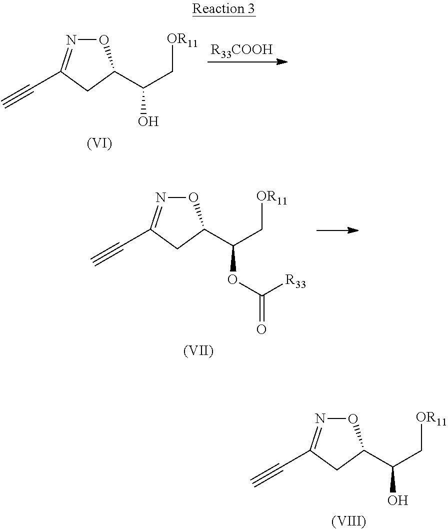

wherein values and alternative values for the variables are as described above for reaction 2 or Structural Formula (III).

In one embodiment, at least 50%, 55%, 60%, 65%, 70%, 75%, 80%, 85%, 90%, 95%, 96%, 97%, 98%, 99%, 99.5%, 99.8%, 99.9% or 100% by weight of the compound obtained by reaction 3 is represented by Structural Formula (VIII).

Reaction 3 can be carried out under commonly known Mitsunobu reaction conditions to form ester (VII), which is later de-esterified to covert the ester into alcohol (VIII).

In one embodiment, for reaction 3 described above, the reacting step is conducted in the presence of an azodicarboxylate (such as diethyl azodicarboxylate (DEAD), di-isopropylazodicarboxylate (DIAD), or di-tert-butyl azodicarboxylate) and triphenylphosphine (TPP). In one embodiment, the azodicarboxylate is di-tert-butyl azodicarboxylate. This step can be carried out in any suitable solvent or solvents. In one embodiment, the reaction is carried out in an organic solvent or solvents, such as tetrahydrofuran (THF), acetonitrile, or toluene.

In one embodiment, for reaction 3 described in any one of the foregoing embodiments, the converting step is conducted with an alcohol in the presence of Zn(OAc)₂ or Cu(OAc)₂. This step can be carried out in any suitable solvent or solvents. In one embodiment, the reaction is carried out in an organic solvent or solvents, such as methanol, tetrahydrofuran (THF), acetonitrile, or toluene.

In one embodiment, for reaction 3 described in any one of the foregoing embodiments, R₃₃COOH can be any carboxylic acid which is suitable for Mitsunobu reaction. In one embodiment, R₃₃ is pyridyl.

In one embodiment, for reaction 3 described in any one of the foregoing embodiments, R₃₃ is 2-pyridyl and R₁₁ is t-butyldiphenylsilyl (TBDPS).

In one embodiment, the present invention is also directed to a method (reaction 4a) of reacting a compound of Structural Formula (VIII) with an epoxide

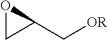

to form a compound of Structural Formula (IXa):

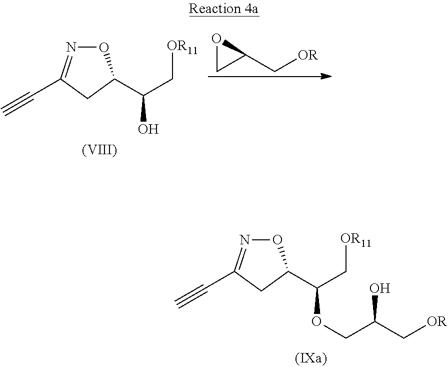

wherein R is —H or a hydroxyl protecting group; and values and alternatives values for the remainder of the variables are as described above for reaction 3.

In one embodiment, at least 45%, 50%, 55%, 60%, 65%, 70%, 75%, 80%, 85%, 90%, 95%, 96%, 97%, 98%, 99%, 99.5%, 99.8%, 99.9% or 100% by weight of the compound obtained by reaction 4a is represented by Structural Formula (IXa).

In another aspect, the present invention is also directed to a method (reaction 4b) of reacting a compound of Structural Formula (VIII) with an epoxide

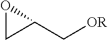

to form a compound of Structural Formula (IXb):

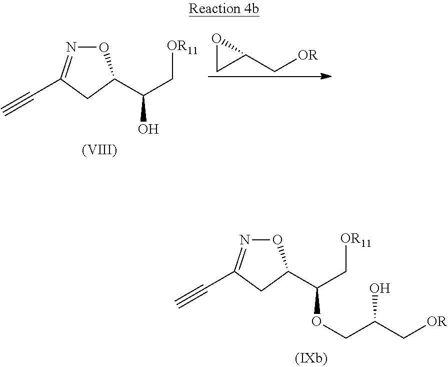

wherein R is —H or a hydroxyl protecting group; and values and alternatives values for the remainder of the variables are as described above for reaction 3.

In one embodiment, at least 45%, 50%, 55%, 60%, 65%, 70%, 75%, 80%, 85%, 90%, 95%, 96%, 97%, 98%, 99%, 99.5%, 99.8%, 99.9% or 100% by weight of the compound obtained by reaction 4b is represented by Structural Formula (IXb).

In one embodiment, for reactions 4a and 4b described above, the reacting step is conducted under commonly known S_{N}2 type ring opening reaction conditions.

In one embodiment, for reactions 4a and 4b described in any one of the foregoing embodiments, this step can be carried out in any suitable solvent or solvents. In one embodiment, the reaction is carried out in an organic solvent or solvents, such as dichloromethane (DCM), ether, tetrahydrofuran (THF), or toluene.

In one embodiment, for reactions 4a and 4b described in any one of the foregoing embodiments, the reacting step is conducted in the presence of (C₁-C ₆) alkyl magnesium halide. In one embodiment, the (C₁-C₆) alkyl magnesium halide is ethylmagnesium bromide.

In one embodiment, for reactions 4a and 4b described in any one of the foregoing embodiments, R is H, optionally substituted methyl, optionally substituted ethyl, or optionally substituted benzyl. In one embodiment, R is benzyl.

In one embodiment, the present invention is also directed to a method (reaction 5a) of converting the compound of Structural Formula (IXa) into a compound of Structural Formula (Xa) in the presence of an amine base.

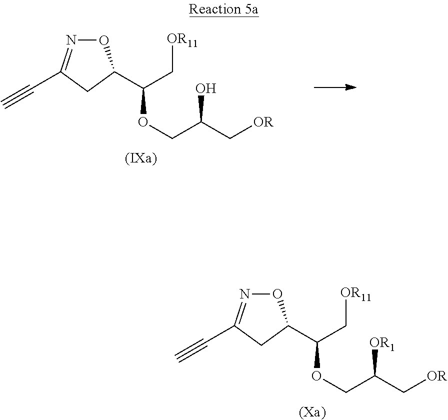

wherein R₁ is a hydroxyl activating group; and values and alternatives values for the remainder of the variables are as described above for reaction 4a.

In one embodiment, at least 50%, 60%, 70%, 80%, 90%, 95%, 96%, 97%, 98%, 99%, 99.5%, 99.8%, 99.9% or 100% by weight of the compound obtained by reaction 5a is represented by Structural Formula (Xa).

In one embodiment, for reaction 5a described above, the amine base is N,N-diisopropylethylamine (DIPEA), triethylamine (TEA), 4-dimethylaminopyridine (DAMP), N-methylmorpholine, 1,4-diazabicyclo[2,2,2]octane (DABCO), 1,5-diazabicyclo[4,3,0]non-5-ene (DBN) , or 1,8-diazabicyclo[5 ,4,0]undec-7-ene (DBU).

In one embodiment, for reaction 5a described in any one of the foregoing embodiments, the reaction is conducted in the presence of 4-dimethylaminopyridine.

In one embodiment, for reaction 5a described in any one of the foregoing embodiments, R₁ is C₁₋₁₀ alkylsulfonate or (C₁-C₁₀) arylsulfonate.

In one embodiment, for reaction 5a described in any one of the foregoing embodiments, R₁ is mesylate, triflate, nonaflate, tresylate, besylate, brosylate, nosylate, or tosylate.

Reaction 5a described in any one of the foregoing embodiments can be carried out in any suitable solvent or solvents. In one embodiment, the reaction is carried out in an organic solvent or solvents, such as dichloromethane (DCM), acetonitrile, or toluene.

In one embodiment, the present invention is also directed to a method (reaction 5b) of converting the compound of Structural Formula (IXb) into a compound of Structural Formula (Xb) in the presence of an acid,

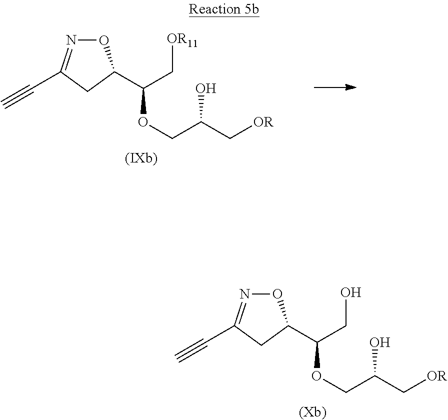

wherein R₁₁ and R are as described above for reaction 4b.

In one embodiment, at least 50%, 60%, 70%, 80%, 90%, 95%, 96%, 97%, 98%, 99%, 99.5%, 99.8%, 99.9% or 100% by weight of the compound obtained by reaction 5b is represented by Structural Formula (Xb).

In one embodiment, for reaction 5b described above, the acid is acetic acid.

In one embodiment, for reaction 5b described in any one of the foregoing embodiments, the converting step is performed in the presence of tetrabutylammonium fluoride (TBAF).

In one embodiment, for reaction 5b described in any one of the foregoing embodiments, R₁₁ is t-butyldiphenylsilyl (TBDPS) and R is benzyl.

Reaction 5b described in any one of the foregoing embodiments can be carried out in any suitable solvent or solvents. In one embodiment, the reaction is carried out in an organic solvent or solvents, such as tetrahydrofuran (THF), dichloromethane (DCM), acetonitrile, or toluene.

The present invention is also directed to a method (reaction 6a) of converting the compound of Structural Formula (Xa) into a compound of Structural Formula (IIa) in the presence of an acid.

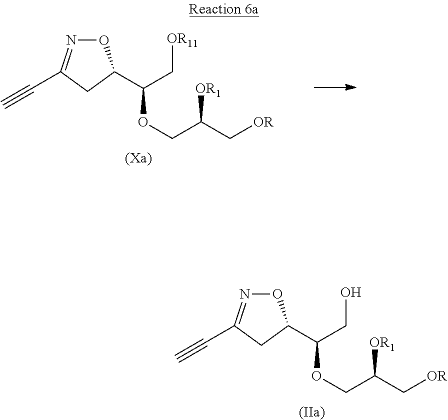

wherein R₁, R₁₁ and R are as described above for reaction 5a. In one embodiment, at least 50%, 60%, 70%, 80%, 90%, 95%, 96%, 97%, 98%, 99%, 99.5%, 99.8%, 99.9% or 100% by weight of the compound obtained by reaction 6a is represented by Structural Formula (IIa).

In one embodiment, for reaction 6a described above, the acid is a carboxylic acid, for example, formic acid, acetic acid, or propionic acid.

In one embodiment, for reaction 6a described in any one of the foregoing embodiments, the converting step is performed in the presence of a de-silylation reagent.

In one embodiment, for reaction 6a described in any one of the foregoing embodiments, the de-silylation reagent is a fluoride salt (e.g., tetrabutylammonium fluoride (TBAF)).

In one embodiment, for reaction 6a described in any one of the foregoing embodiments, R₁₁ is t-butyldiphenylsilyl (TBDPS).

Reaction 6a described in any one of the foregoing embodiments can be carried out in any suitable solvent or solvents. In one embodiment, the reaction is carried out in an organic solvent or solvents, such as tetrahydrofuran (THF), dichloromethane (DCM), acetonitrile, or toluene.

The present invention is also directed to a method (reaction 6b) of converting the compound of Structural Formula (Xb) into a compound of Structural Formula (IIb).

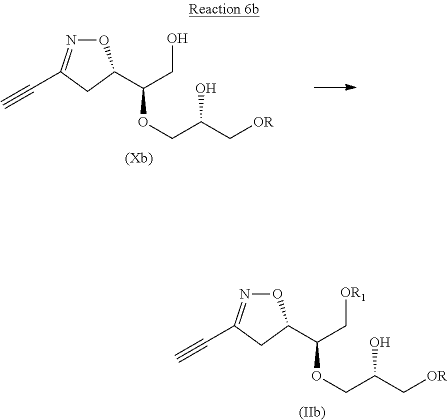

wherein R₁ is a hydroxyl activating group; R is as described above for reaction 5b.

In one embodiment, at least 50%, 60%, 70%, 80%, 90%, 95%, 96%, 97%, 98%, 99%, 99.5%, 99.8%, 99.9% or 100% by weight of the compound obtained by reaction 6b is represented by Structural Formula (IIb).

In one embodiment, for reaction 6b described above, R₁ is (C₁-C₁₀) alkylsulfonate or (C₁-C₁₀) arylsulfonate. In another embodiment, R₁ is tosylate, besylate, brosylate, nosylate, mesylate, tresylate, nonaflate and triflate.

Reaction 6b described in any one of the foregoing embodiments can be carried out in any suitable solvent or solvents. In one embodiment, the reaction is carried out in an organic solvent or solvents, such as dichloromethane (DCM), acetonitrile, or toluene. Alternatively, reaction 6b can be carried out in the presence of water. In one embodiment, the reaction is carried out in a mixture of water and an organic solvent, such as dichloromethane (DCM) and water.

In one embodiment, the present invention is also directed to a method (reaction 7) of cyclizing a compound of Structural Formula (IIa) or (IIb) in the presence of a strong base to form a compound of Structural Formula (I).

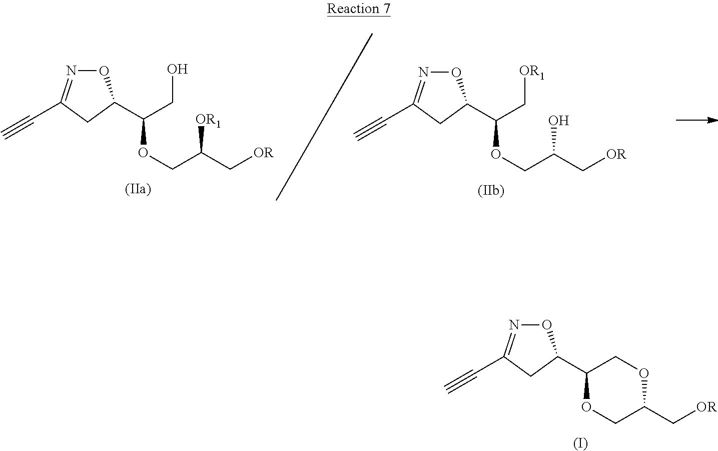

wherein R is —H or a hydroxyl protecting group; and R₁ is a hydroxyl activating group.

In one embodiment, at least 45%, 50%, 55%, 60%, 65%, 70%, 75%, 80%, 85%, 90%, 95%, 96%, 97%, 98%, 99%, 99.5%, 99.8%, 99.9% or 100% by weight of the compound obtained by reaction 7 is represented by Structural Formula (I).

In one embodiment, for reaction 7 described above, R is H, optionally substituted methyl, optionally substituted ethyl, or optionally substituted benzyl. In one embodiment, R is benzyl.

In one embodiment, for reaction 7 described in any one of the foregoing embodiments, R₁ is mesylate, triflate, nonaflate, tresylate, besylate, nosylate, brosylate, or tosylate.

In one embodiment, for reaction 7 described in any one of the foregoing embodiments, the strong base is an alkali metal hydroxide (e.g., NaOH, KOH), or an alkali metal C₁₋₆alkoxide (e.g., NaOMe, KO^{t}Bu), or an alkali metal bis(trimethylsilyl)amide. In one embodiment, the strong base is sodium tert-butoxide.

In one embodiment, for reaction 7 described in any one of the foregoing embodiments, R is benzyl and R₁ is tosylate.

Reaction 7 described in any one of the foregoing embodiments can be carried out in any suitable solvent or solvents. In one embodiment, the reaction is carried out in an organic solvent or solvents, such as tetrahydrofuran (THF), dichloromethane (DCM), dimethylformamide (DMF), acetonitrile, toluene, or dimethyl sulfoxide (DMSO). Alternatively, when the base is compatible with aqueous condition, reaction 7 can be carried out in the presence of water. In one embodiment, the reaction is carried out in a mixture of water and an organic solvent, such as those described above. When the organic solvent is not miscible with water, a phase transfer catalyst, such as a quaternary ammonium salt (e.g., tetrabutylammonium chloride, bromide or iodide) can be used.

The present invention is also directed to a compound represented by Structural Formula (I) as described above.

In one embodiment, for the compound represented by Structural Formula (I), R is H, optionally substituted methyl, optionally substituted ethyl, or optionally substituted benzyl.

In one embodiment, for the compound represented by Structural Formula (I), R is benzyl.

The present invention is also directed to a compound represented by Structural Formula (III) as described above.

In one embodiment, for the compound represented by Structural Formula (III), R₁₁ is t-butyldimethylsilyl (TBDMS), t-butyldiphenylsilyl (TBDPS), or trityl.

In one embodiment, for the compound represented by Structural Formula (III) described in any one of the foregoing embodiments, R₂₂ is trimethylsilyl (TMS), triethylsilyl (TES), t-butyldimethylsilyl (TBDMS), dimethylthexylsilyl, biphenyldimethylsilyl, triisopropylsilyl, biphenyldiisoporpylsilyl, or 2-(2-hydroxypropyl).

In one embodiment, for the compound represented by Structural Formula (III) described in any one of the foregoing embodiments, R₁₁ is t-butyldiphenylsilyl (TBDPS) and R₂₂ is trimethylsilyl (TMS).

When a compound is designated by a name or structure that indicates a single enantiomer, unless indicated otherwise, the compound is at least 45%, 50%, 55%, 60%, 65%, 70%, 75%, 80%, 85%, 90%, 95%, 96%, 97%, 97.5%, 98%, 98.5%, 99%, 99.5%, 99.8%, 99.9% or 100% optically pure (also referred to as “enantiomerically pure”). Optical purity is the weight in the mixture of the named or depicted enantiomer divided by the total weight in the mixture of both enantiomers. Alternatively, when a compound is designated by a name or structure that indicates a single enantiomer, unless indicated otherwise, the compound has a percent enantiomeric excess of at least 20%, 40%, 60%, 80%, 90%, 92%, 94%, 95%, 96%, 97%, 97.5%, 98%, 98.5%, 99%, 99.5%, 99.8%, 99.9% or 100%. Percent enantiomeric excess, or percent e.e., is the difference between the percent of the named or depicted enantiomer and the opposite enantiomer.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 45%, 50%, 55%, 60%, 65%, 70%, 75%, 80%, 85%, 90%, 95%, 96%, 97%, 98%, 99%, 99.5%, 99.8%, 99.9% or 100% by weight. The stereoisomeric purity in this case is determined by dividing the total weight in the mixture of the stereoisomers encompassed by the name or structure by the total weight in the mixture of all of the stereoisomers.

The term “halide” as used herein refers to chloride, bromide, and iodide.

The term “alkyl” as used herein refers to saturated straight-chain or branched aliphatic group. As used herein, a (C₁-C₆) alkyl group containing one to six carbon atoms.

An “aliphatic group” is acyclic, non-aromatic, consists solely of carbon and hydrogen and may optionally contain one or more units of unsaturation, e.g., double and/or triple bonds. An aliphatic group may be straight chained or branched. An aliphatic group typically contains between about one and about twenty carbon atoms, typically between about one and about ten carbon atoms, more typically between about one and about six carbon atoms. A “substituted aliphatic group” is substituted at any one or more “substitutable carbon atoms.” A “substitutable carbon atom” in an aliphatic group is a carbon in the aliphatic group that is bonded to one or more hydrogen atoms. One or more hydrogen atoms can be optionally replaced with a suitable substituent group.

The term “aryl” used herein refers to a carbocyclic aromatic ring. The term “aryl” may be used interchangeably with the terms “aryl ring” “aromatic ring” and “carbocyclic aromatic ring.” An aryl group typically has six to fourteen ring atoms. Examples includes phenyl, naphthyl, anthracenyl, 1,2-dihydronaphthyl, 1,2,3,4-tetrahydronaphthyl, fluorenyl, indanyl, indenyl and the like. A “substituted aryl group” is substituted at any one or more substitutable ring atom, which is a ring carbon atom bonded to a hydrogen.

Unless stated otherwise, exemplary organic solvents include, but are not limited to, ethereal solvents (e.g., diethyl ether, methyl tert-butyl ether, tetrahydrofuran, 1,4-dioxane and dimethoxyethane), aromatic solvents (e.g., benzene and toluene), chlorinated solvents (e.g., methylene chloride and 1,2-dichloroethane), alcohol solvents (e.g., methanol, ethanol, isopropanol), dimethylformamide, dimethyl sulfoxide and acetonitrile.

Unless stated otherwise, exemplary base includes, but are not limited to alkali metal C₁₋₆ alkoxide (e.g., NaOMe, KO^{t}Bu), alkali metal hydroxide (e.g., NaOH, KOH), alkali metal carbonate (e.g., Na₂CO₃, K₂CO₃ or Cs₂CO₃), amine (e.g., ethylamine, propylamine, dimethylamine, trimethylamine, isopropyethylamine, pyridine), ammonia, alkali metal fluoride (e.g., NaF or KF), and alkali metal phosphate (e.g., Na₃PO₄, Na₂HPO₄, NaH₂PO₄, K₂HPO₄, KH₂PO₄ or K₃PO₄).

### General Synthetic Schemes

Compounds of the invention may be prepared using the synthetic transformations illustrated in Scheme I.

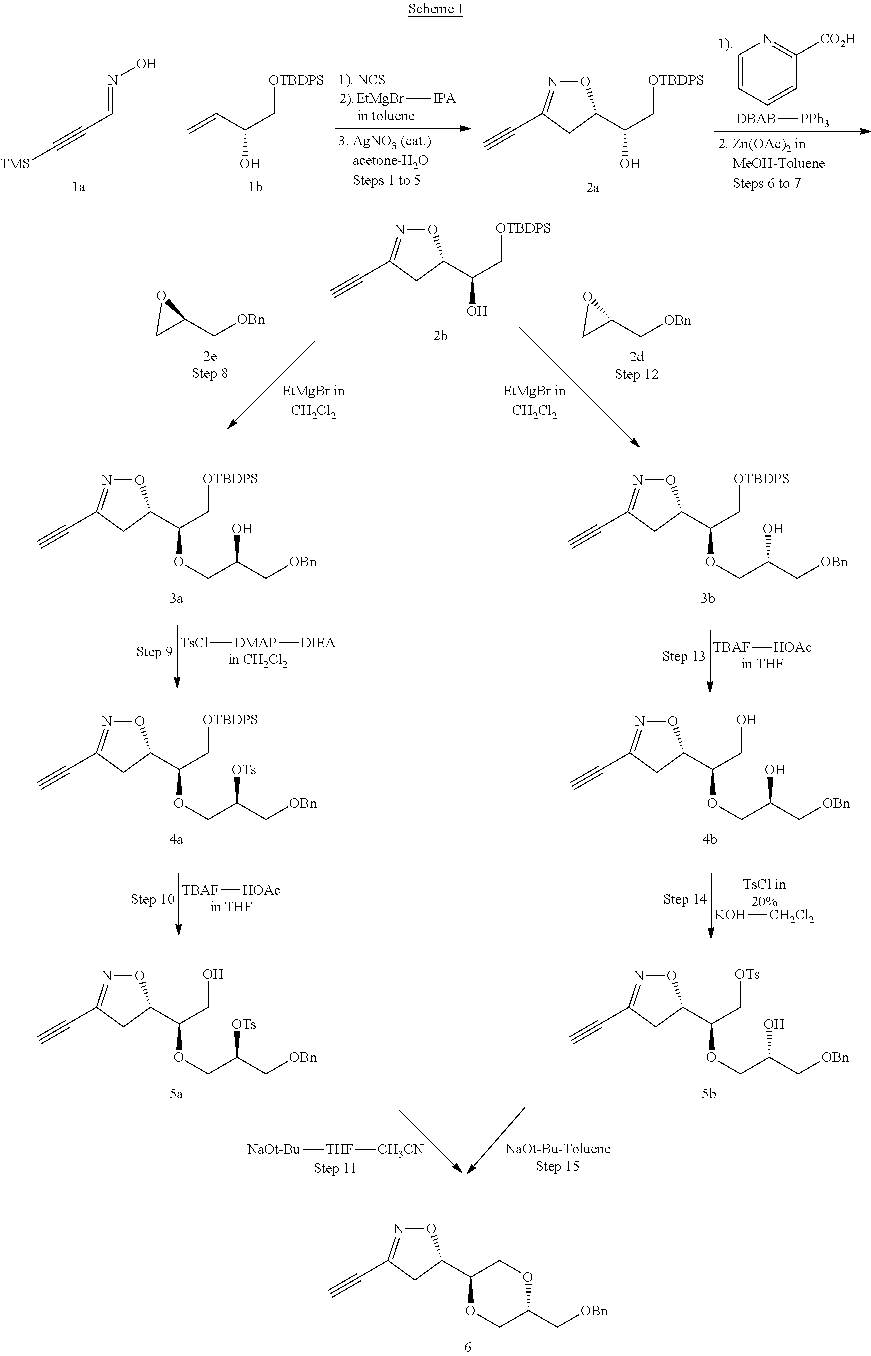

The following examples are ordered according to the final general procedure used in their preparation. The synthetic routes to any novel intermediates are detailed by sequentially listing the general procedure (letter codes) in parentheses after their name with additional reactants or reagents as appropriate.

### LIST OF ABBREVIATIONS

ACN Acetonitrile
CO Carbon monoxide
d doublet
DBU 1,8-Diazabicyclo[5.4.0]undec-7-ene
dd doublet of doublet
DCM Dichloromethane (methylene chloride)
DIEA N,N-diisopropylethylamine
DMF N,N-dimethylformamide
DMSO Dimethyl sulphoxide
DMAP 4-Dimethylaminopyridine
DMA Dimethylacetamide
equiv. Equivalent(s)
EtOAc Ethyl acetate
EtOH Ethanol
g Gram(s)
h Hour(s)
IPA Isopropyl alcohol
KOAc Potassium Acetate
KOBt Potassium t-butoxide
m multiplet
MeOH Methyl alcohol
min Minute(s)

M Molarity
Mmol millimol
mp Melting point
N Normality
NaOtBu Sodium t-butoxide
NLT No less than
NMT No more than
Rac Racemic
R_{f} Retention Factor (TLC)
T_{R} HPLC Retention Time
RT Room temperature
s singlet
t triplet
TBAF Tetrabutyl ammonium fluoride
TBDPS -Cl Tert-butyldiphenylsilyl chloride
TEA Triethyl amine
TFA Trifluoroacetic acid
THF Tetrahydrofuran
Temp Temperature

Preparations and Examples
Exemplification

### EXAMPLE #1

### Step 1: (E/Z)-3-(Trimethylsilyl)propiolaldehyde oxime (1a)

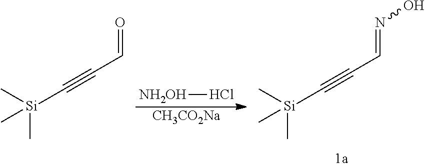

A 500-mL round-bottom flask was charged hydroxylamine hydrochloride (10.92 g, 154 mmol), sodium acetate (23.12 g, 279 mmol), and methanol (283 mL). A white suspension resulted. To this mixture was added 3-(trimethylsilyl)propiolaldehyde (18.12 g, 139 mmol). The reaction mixture was stirred at 20° C. for 1.5 h. A sample was taken, and analyzed for reaction completion by HPLC. The reaction mixture was filtered to remove solids. The filtrate was concentrated in vacuo to approximately 100 mL volume. The concentrate was diluted with t-butyl methyl ether (250 mL). 6% of NaHCO₃ aqueous solution (200 mL) was added slowly. The mixture was stirred at 25° C. for NLT 30 min and allowed to settle for NLT 15 min. The upper organic layer was dried with Na₂SO₄, filtered, and rinsed with t-butyl methyl ether (30 mL). The combined filtrate was concentrated in vacuo to yield brown oil (20.05 g). The oil product was used directly to next step without further purification, ¹H-NMR in CDCl₃ δ E-isomer: 0.22 (9H, s), 7.35 (1H, s); Z-isomer: 0.24 (9H, s), 6.77 (1H, s).

### Step 2: (E/Z)-N-Hydroxy-3-(trimethylsilyl)propiolimidoyl chloride

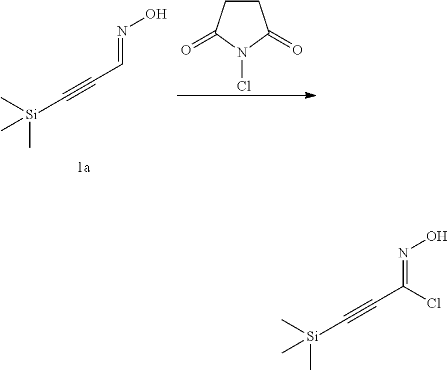

A 1-L round-bottom flask was charged with (E/Z)-3-(trimethylsilyl)propiolaldehydeoxime (20.05 g, 131 mmol), DCM (440 mL), and N-chlorosuccinimide (19.68 g, 144 mmol). The reaction was stirred at RT for NLT 3 h. A sample was taken, and analyzed for reaction completion by HPLC. The solvent was removed in vacuo to yield an orange slurry. The residue was diluted with diethyl ether (200 mL). The resulting mixture was cooled to ˜0° C., and stirred for 2 h. The solid (succinimide) was filtered off. The product solution was concentrated in vacuo to yield orange oil (21.57 g), which was used in Step 4 without further purification.

### Step 3: (R)-1-(tert-butyldiphenylsilyloxy)but-3-en-2-ol (1b)

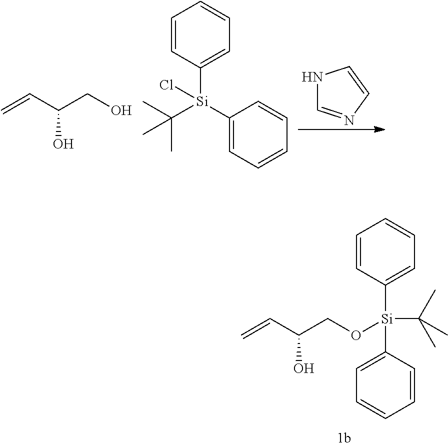

A 1-L three-neck, round-bottom flask was charged (R)-but-3-ene-1, 2-diol (20.29 g, 230 mmol), DCM (570 mL), and imidazole (15.85 g, 230 mmol). The temperature was reduced to about 0° C. tert-butylchlorodiphenylsilane (61.2 mL, 231 mmol) was added slowly while maintaining the internal temperature of NMT 5° C. The reaction mixture was stirred for 15 min at ˜0° C., and allowed to rise to 20° C. over 2 h. The reaction mixture was stirred at 20° C. for NLT 4 h. A sample was taken, and analyzed for reaction completion by HPLC. The reaction mixture was filtered to remove solids, and then the filtrate washed twice with 10% citric acid (150 mL). The final organic layer was dried with MgSO₄, filtered and rinsed with DCM (30 mL). The filtrate was concentrated in vacuo to give (R)-1-(tert-butyldiphenylsilyloxy)but-3-en-2-ol (81.75 g, ee=99.5%) as a slightly colored oil (81.75 g). The product was used directly to next step without further purification. ¹HNMR in CDCl₃: δ1.13 (9H, s), 2.65 (1H, s, br), 3.59 (1H, dd, J=10.1, 7.5 Hz), 3.74 (1H, dd, J=10.1, 3.7 Hz), 4.29 (1H, m), 5.21 (1H, dt, J=10.5, 1.5 Hz), 5.36 (1H, dt, J=10.5, 1.5 Hz), 5.83 (1H, ddd, J=17.2, 10.5, 5.6 Hz), 7.45 (6H, m), 7.71 (4H, m).

### Step 4: (S)-2-(tert-butyldiphenylsilyloxy)-1-(S)-3-((trimethylsilyl)ethynyl)-4,5-dihydroisoxazol-5-yl)ethanol (2a)

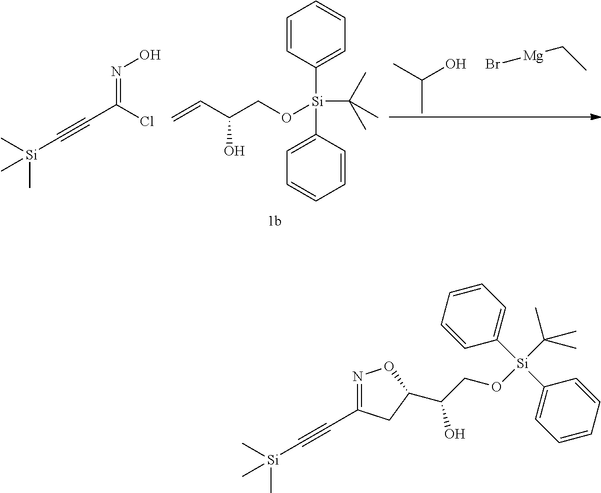

To a reaction vessel were added (R)-1-(tert-butyldiphenylsilyloxy)but-3-en-2-ol (1b) (3.2 g, 9.33 mmol, 1 equiv.) and toluene (40 mL). The solution was then cooled to −10° C. Propan-2-ol (2.50 mL, 1.97 g, 32.7 mmol, 3.5 equiv.) was added at the internal temperature of NMT 0° C. 9.33 mL of 3.0 M ethylmagnesium bromide (28.0 mmol, 3.0 equiv.) in diethyl ether was added dropwise while keeping the internal temperature below 0° C. The mixture was stirred at 0° C. for 15 min before been used in the 2+3 cyclization. To the above mixture at 0° C. was slowly added a solution of (E/Z)-N-hydroxy-3-(trimethylsilyl)propiolimidoyl chloride (2.82 g, 15.88 mmol, 1.7 equiv.) in toluene (20 mL) via a syringe pump over 3 h. The reaction mixture was stirred at 0° C. for NLT 2 h. The reaction progress was monitored by HPLC over time. The reaction mixture was quenched by addition of 96 mL of 5% citric acid at <20° C. The reaction mixture was stirred at 20° C. for 1 h. The mixture was filtered through a bed of filter aid to remove insoluble in the mixture, and rinsed with toluene (5 mL). The upper organic layer was separated, and washed with 48 mL of 25% brine. The organic was dried over MgSO₄, filtered through a bed of filter aid, and rinsed with toluene (10 mL). The filtrate was concentration under vacuum to give an oil, which was then purified by a silica gel column eluting with heptane/EtOAc (4:1 R_{f}=0.5) to afford 3.45 g of the product: The diastereomeric ratio is ˜96.5:3.5 by HPLC 1; ¹HNMR in CDCl₃: δ0.24 (9H, s), 1.07 (9H, s), 2.23 (1H, d, J=6.6 Hz), 3.06 (1H, d, J=1.8 Hz), 3.08 (1H, d, J=3.0 Hz), 3.73 (3H, m), 4.83 (1H, ddd, J=10.3, 9.1, 3.6 Hz), 7.41 (6H, m), 7.65 (4H, m); ¹³CNMR in CDCl₃: δ19.7, 27.1, 39.9, 64.3, 72.6, 81.2, 92.5, 104.4, 127.5, 129.6, 132.5, 135.4, 143.0.

HPLC 1 Conditions: Column: Unison UK-C₁₈, 150×4.6, column temperature: 30C; Mobile phase A: 100% H₂O with 0.1% HClO₄, Mobile phase B: 100% CH₃CN.

| Time (min) | A % | B % |
|---|---|---|
| 0 | 80 | 20 |
| 10 | 0 | 100 |
| 15 | 0 | 100 |
| 15.10 | 80 | 20 |

Postrun: 5 min

### Step 5: (S)-2-(tert-butyldiphenylsilyloxy)-1-((S)-3-ethynyl-4,5-dihydroisoxazol-5-yl)ethanol (2a)

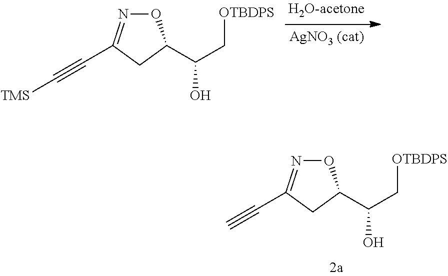

A 2 L three neck flask was charged with silver nitrate (1.103 g, 0.0065 mol, 10 mol %), water (117 g, 6.5 mol, 100 equiv.) and (S)-2-(tert-butyldiphenylsilyloxy)-1-((S)-3-((trimethylsilyl)ethynyl)-4,5-dihydroisoxazol-5-yl)ethanol (30.25 g, 0.0650 mol, 1 equiv.). Acetone (1200 mL, 40 vol) was charged to the reactant mixture to form a cloudy solution. The reaction was mixed at RT without light overnight. A sample was taken, and analyzed for reaction completion by HPLC. The reaction mixture was concentrated under vacuum to ˜180 mL volume to give heterogeneous oil. EtOAc (300 mL) and water (300 mL) were charged to this product oil, mixing for NLT 15 min to give a biphasic suspension. The suspension was filtered to remove insoluble silver related solid. The upper organic layer was separated. The lower aqueous layer was back extracted with EtOAc (150 mL). The combined organic was washed with 25% brine (250 mL), and concentrated to dryness to give the crude product as a yellow oil. The crude product was purified by silica gel column, eluting with EtOAc:Hexane 1:4 (v/v). The product's fractions were pooled together, and concentrated to dryness to give an off-white oil (21.0 g, 82%), solidified on standing: mp: 60° C. (uncorrected); MS-ESI: 394 (M+1); ¹HNMR in CDCl₃: δ1.07 (9H, s), 2.26 (1H, s, br), 3.08 (1H, s), 3.10 (1H, d, J=1.4 Hz), 3.36 (1H, s), 3.70 (1H, s, br), 3.75 (2H, m), 4.85 (1H, td, J=9.6, 3.6 Hz), 7.40 (6H, m), 7.65 (4H, m).

### Step 6: (R)-2-(tert-butyldiphenylsilyloxy)-1-((S)-3-ethynyl-4,5-dihydroisoxazol-5-yl)ethyl picolinate

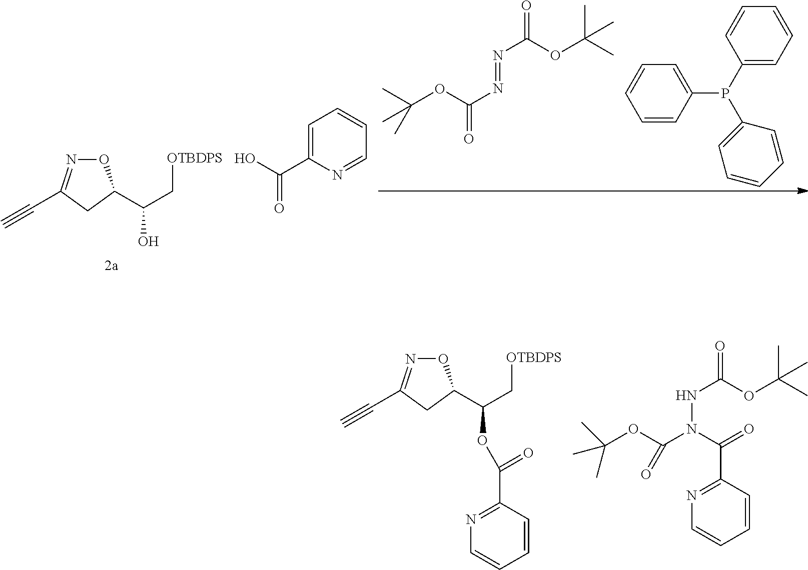

To a 500 mL three-neck round bottom flask equipped with a mechanical stirrer were charged: (S)-2-(tert-butyldiphenylsilyloxy)-1-((S)-3-ethynyl-4,5-dihydroisoxazol-5-yl)ethanol (2a) (5.15 g, 13.0 mmol, 1.00 equiv.), 2-picolinic acid (4.90 g, 50.5 mmol, 3.9 equiv.), triphenylphosphine (5.22 g, 19.9 mmol, 1.5 equiv.), and toluene (100 mL, KF=74 ppm). The resulting mixture was stirred and adjusted to an internal temperature of ˜20° C. The mixture was stirred for 15 min under N₂. A solution of di-tert-butyl azodicarboxylate (4.58 g, 19.9 mmol, 1.5 equiv.) in toluene (17.0 mL) was added at an internal temperature of NMT 35° C. (slightly exothermic), and rinsed with toluene (3.0 mL). The reaction mixture was adjusted to 30° C., and agitated under N₂ at 30° C. for NLT 4 h (note: cloudy mixture became a clear solution after 1 h). A sample was taken, and analyzed for reaction completion by HPLC. The reaction mixture was cooled to ˜20° C. 100 g of 5% NaHCO₃ aqueous solution was added slowly to the reaction mixture to quench the reaction. The mixture was stirred for NLT 15 min, and allowed to settle for NLT 15 min at 25° C. The upper organic layer was separated and concentrated to ˜½ of the original volume (-60 mL). The solution was adjusted to the internal temperature of NLT 30° C., and heptane (50.0 mL) added. The solution was adjusted to 30° C. A solution (110 g) of H₃PO₄ in water and DMF prepared from 31.0 g of 85% H₃PO₄ in water (60.0 g) and DMF (19.0 g), was added to the above product solution (toluene: heptane 1:1). The mixture was stirred, and the internal temperature adjusted to about 30° C. The reaction mixture was stirred for NLT 2 h to destroy the reaction by-product, di-tert-butyl-1-picolinoylhydrazine-1,2-dicarboxylate. The reaction mixture was slowly cooled to ˜10° C., and stirred for 1 h more. Triphenylphosphine oxide was removed by filtration, and washed with heptane-toluene (10 mL, 1:1). The filtrate was adjusted to the internal temperature of NLT 25° C. The mixture was allowed to settle, and the upper organic phase separated. The organic was washed with 5% NaHCO₃ (100 g), and 25% brine (50 g) at 25° C. respectively. The organic was dried over MgSO₄, filtered, and rinsed with toluene (5 mL). The organic was concentrated to ˜20 mL volume, purified by a silica gel column eluting gradient from 100% heptane to 55% heptane −45% EtOAc. The product's fraction were pooled together and concentrated to dryness to give a slight-brown oil (6.02 g, potency=92.9%, 90% isolated yield). MS-ESI: 499 (M+1) ; ¹HNMR in CDCl₃ δ1.03 (s, 9H), 3.19 (m, 2H), 3.36 (s, 1H), 3.94 (dd, J=11.3, 4.5 Hz, 1H), 4.02 (dd, J=11.3, 4.5 Hz, 1H), 5.16 (ddd, J=10.7, 7.9, 5.6 Hz, 1H), 5.41 (q, J=5.0 Hz, 1H), 7.26 (m, 2H), 7.3-7.40 (m, 4H), 7.48 (ddd, J=7.6, 4.7, 1.0 Hz, 1H), 7.62 (m, 4H), 7.82 (td, J=7.6, 1.8 Hz, 1H), 8.02 (dt, J=7.6, 1.0 Hz, 1H), 8.78 (ddd, J=4.7, 1.8, 1.0 Hz, 1H).

### Step 7: (R)-2-(tert-butyldiphenylsilyloxy)-1-((S)-3-ethynyl-4,5-dihydroisoxazol-5-yl)ethanol (2b)

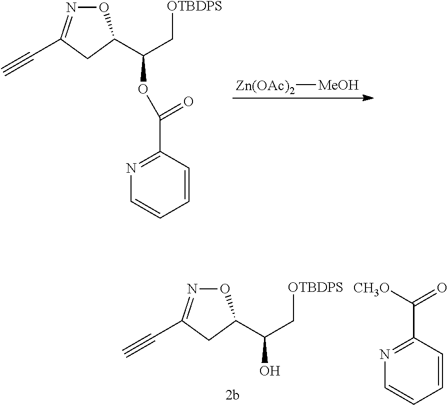

A 250 mL round bottom flask equipped with a mechanical stirrer was charged with (R)-2-((tert-butyldiphenylsilyl)oxy)-1-((S)-3-ethynyl-4,5-dihydroisoxazol-5-yl)ethyl picolinate (6.00 g, potency=92.9% by ¹HNMR, 11.2 mmol, 1.0 equiv.), toluene (50 mL), methanol (2.5 mL, 61.6 mmol, 5.5 equiv.), and zinc acetate dihydrate (2.51 g, 11.2 mmol, 1.0 equiv.). The near colorless reaction solution was stirred at 20° C. for NLT 2 h. A sample was taken, and analyzed by HPLC for reaction completion. The reaction mixture was diluted with heptane (50 mL). The mixture was then washed with 10% citric acid (100 mL×3) to remove methyl-2-picolinate, as monitored by HPLC), and 25% brine (100 mL). The organic was dried over MgSO₄ (3.0 g), filtered and rinsed with toluene (5 mL). The filtrate was concentrated to dryness at 50° C. under vacuum to give (R)-2-(tert-butyldiphenylsilyloxy)-1-((S)-3-ethynyl-4,5-dihydroisoxazol-5-yl)ethanol as a slightly brown oil (4.60 g, potency=90.6% by ¹HNMR, potency adjusted yield=94.5%), solidified on standing to give a waxy solid. This semi-solid was used directly to next step without any further purification. However, an analytical standard sample was obtained by triturating this waxy solid with hexane: mp: 60-62° C.; MS-ESI: 394 (M+1); ¹HNMR in CDCl₃: δ1.07 (9H, s), 2.40 (1H, d, J=3.9 Hz, OH), 3.04 (1H, dd, J=17.3, 11.0 Hz), 3.18 (1H, dd, J=17.3, 7.7 Hz), 3.34 (1H, s), 3.76 (3H, m), 4.76 (1H, m), 7.43 (6H, m), 7.64 (4H, m).

### Step 8: (4S,7R)-7-((S)-3-ethynyl-4,5-dihydroisoxazol-5-yl)-11,11-dimethyl-1,10,10-triphenyl-2,6,9-trioxa-10-siladodecan-4-ol (3a)

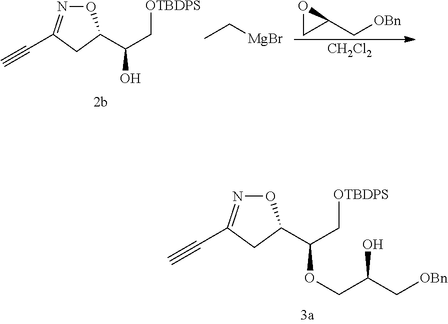

To a 40 mL vial were added (R)-2-(tert-butyldiphenylsilyloxy)-1-((S)-3-ethynyl-4,5-dihydroisoxazol-5-yl)ethanol (1.71 g, potency=95.3%, 4.1 mmol, 1.0 equiv.), and CH₂Cl₂ (12 mL). The solution was stirred at RT for 5 min. The clear solution was cooled to about -5° C. 1.38 mL (4.1 mmol, 1.0 equiv.) of 3.0 M ethylmagnesium bromide solution in ether was added slowly at <5° C. The resulting light brown solution was stirred at ˜0° C. for 15 min, and the temperature allowed to rise to RT. 1.65 g (10.0 mmol, 2.5 equiv.) of benzyl-R-glycidyl ether was added slowly. The reaction mixture was stirred at 20+/−5° C. for 15 min and heated at 55° C. in oil bath for 20 h (internal temp: 53° C.). The solution was cooled to RT, and a sample taken to measure reaction completion (-92% conversion). The reaction mixture was diluted with 5 mL CH₂Cl₂, and quenched with 10% citric acid aqueous solution (12 mL). The lower organic layer was then washed with 10% citric acid solution (12 mL), 20% brine (12 mL), dried over MgSO₄, filtered, and rinsed with CH₂Cl₂ (5 mL). The organic layer was concentrated to dryness. The crude product was taken into 3 mL of toluene, and purified with a silica gel column, eluting with 100% heptane to 70% heptane-30% ethyl acetate. The products fractions were pooled together and concentrated to dryness to give 2.28 g of the product, potency by ¹HNMR=70.0 wt %, potency adjusted yield=70% after isolation). MS-ESI: 558 (M+1); ¹HNMR in CDCl3: δ 1.10 (9H, s), 2.56 (1H, d, J=4.7 Hz), 2.93 (1H, dd, J=16.8, 11.2 Hz), 3.15 (1H, dd, J=16.8, 8.0 Hz), 3.37 (1H, s), 3.4-3.9 (7H, m), 4.5-4.6 (3H, m), 4.91 (1H, ddd, J=11.7, 8.3, 3.4 Hz) 7.3-7.7 (15H, m).

### Step 9: (4S,7R)-7-((S)-3-ethynyl-4,5-dihydroisoxazol-5-yl)-11,11-dimethyl-1,10,10-triphenyl-2,6,9-trioxa-10-siladodecan-4-yl 4-methylbenzenesulfonate (4a)

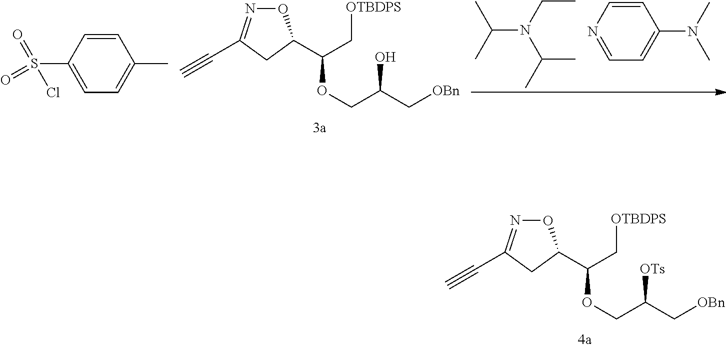

A 500 mL three-neck round bottom flask was charged with DMAP (2.18 g, 17.9 mmol, 1.0 equiv.), diisopropylethylamine (6.93 g, 53.7 mmol, 3.0 equiv.), and (4S,7R)-7-((S)-3-ethynyl-4,5-dihydroisoxazol-5-yl)-11,11-dimethyl-1,10,10-triphenyl-2,6,9-trioxa-10-siladodecan-4-ol (3a) (13.60 g, potency=73.0, 17.8 mmol) in CH₂Cl₂ (125 mL). The mixture was stirred at RT for 5 min p-Toleuenesulfonyl chloride (6.95 g, 36. 5 mmol, 2.0 equiv.) was added all at once. The reaction mixture was stirred at RT for 5 min , and heated to reflux for NLT 24 h. A sample was pulled for analysis to measure reaction completion. 5% NaHCO₃ aqueous solution (100 mL) was added. The mixture was stirred at 25° C. for NLT 15 min, and allowed to settle for NLT 15 min The lower organic phase was separated (clear phase separation). The organic layer was washed with 5% NaHCO₃ (100 mL), 20% brine (100 mL). The organic layer was dried over MgSO₄, filtered, and rinsed with CH₂Cl₂ (20 mL). The organic layer was concentrated to ˜dryness. The crude product was dissolved in toluene (15 mL), purified with a silica gel column eluting with 100% heptane to 70% heptane-30% EtOAc. The product's fractions were pooled together and concentrated to dryness to give 17.20 g of the product (potency=68.1wt %, 92% isolated yield). MS-ESI: 712 (M+1); ¹HNMR in CDCl₃: δ1.08 (9H, s), 2.39 (3H, s), 2.88 (1H, dd, J=17.1, 11.3 Hz), 3.02 (1H, dd, J=17.1, 8.3 Hz), 3.36 (1H, s), 3.55-3.85 (10H, m), 4.42 (3H, m), 4.68 (1H, m), 4.83 (1H, ddd, J=11.6, 8.3, 3.4 Hz), 7.3-7.7 (19 H, m).

### Step 10: (S)-1-(benzyloxy)-3-((R)-1-((S)-3-ethynyl-4,5-dihydroisoxazol-5-yl)-2-hydroxyethoxy)propan-2-yl 4-methylbenzenesulfonate (5a)

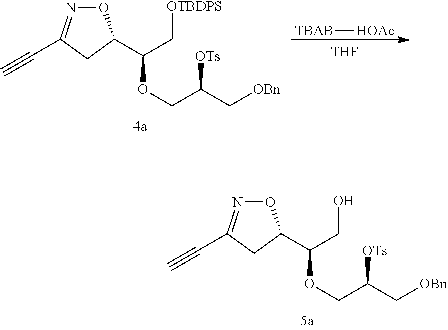

A 500 mL round bottom flask was charged with (4S,7R)-7-((S)-3-ethynyl-4,5-dihydroisoxazol-5-yl)-11,11-dimethyl-1,10,10-triphenyl-2,6,9-trioxa-10-siladodecan-4-yl 4-methylbenzenesulfonate (4a) (17.05 g, potency=68.1%, 16 3 mmol) in THF (100 mL), and acetic acid (1.47 g, 24.5 mmol, 1.5 equiv.). The mixture was stirred at 5 min or until all of the oil dissolved. The solution was cooled to 0° C. 20.39 mL (20.4 mmol, 1.3 equiv.) of 1M TBAF solution in THF was added at the internal temperature of <10° C. The solution was stirred at 0° C. for 5 min., and adjusted to 20° C. over 45 min The solution was stirred at 20° C. for NLT 1 h. A sample was taken and analyzed for reaction completion by HPLC. The solution was concentrated to −30 mL volume. DCM (105 mL) was added. The mixture was washed with 5% NaHCO₃ aqueous solution (100 mL×2). The organic layer was dried over MgSO₄, filtered, and rinsed with CH₂Cl₂ (10 mL). The filtrate was concentrated to dryness. The crude product was dissolved in toluene (10 mL), and purified by a silica gel column, eluting with 100% heptane to 50% heptane-50% acetone. The product fractions were pooled together, and concentrated to dryness to give an oil (7.72 g, potency=92.4 wt %, isolated yield=92.0%): MS-ESI: 474 (M+1); ¹HNMR in CDCl₃ δ2.25 (1H, t, J=6.6 Hz, OH), 2.42 (3H, s), 2.97 (1H, dd, J=17.2, 11.1 Hz), 3.07 (1H, dd, J=17.2, 8.5 Hz), 3.35 (1H, s), 3.48-3.62 (4H, m), 3.68 (1H, m), 3.80 (1H, dd, J=11.1, 7.3, 3.8 Hz), 3.87 (1H, dd, J=11.1, 4.2 Hz), 4.42 (2H, q, J=11.9 Hz), 4.68 (1H, ddd, J=11.1,7.3, 3.8 Hz), 4.73 (1H, m), 7.21 (2H, m), 7.29 (5H, m), 7.76 (2H, m).

### Step 11: (S)-5-((2R,5R)-5-(benzyloxymethyl)-1,4-dioxan-2-yl)-3-ethynyl-4,5-dihydroisoxazole (6)

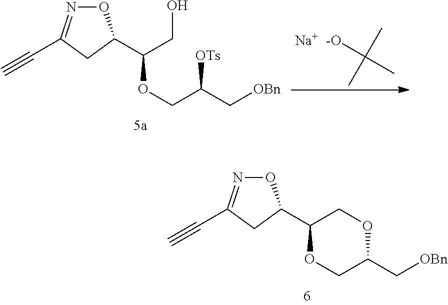

To a 100 mL round bottom flask were added (S)-1-(benzyloxy)-3-((R)-1-((S)-3-ethynyl-4,5-dihydroisoxazol-5-yl)-2-hydroxyethoxy)propan-2-yl-4-methylbenzene sulfonate (5a) (680 mg, potency=92.0%, 1.3 mmol) and ACN (10 mL, KF=6 ppm). The solution was stirred at RT for 10 min to achieve a clear solution. The solution was cooled to 0° C. A filtered solution of sodium tert-butoxide (140 mg, 97%, 1 4 mmol, ˜1.0 equiv.) in THF (2.0 mL) was added slowly into the reaction mixture at 0-5° C. The light brown reaction mixture was stirred at 0-5° C. for NLT 6 h, and allowed to rise to RT. The reaction mixture was stirred at RT for 8 h more. 5% NaHCO₃ aqueous. solution (2.0 g) was added to the reaction mixture to quench it. The reaction mixture was concentrated to dryness to ˜2 mL volume, and toluene (10 mL) added. The mixture was washed with 5% NaHCO₃ aqueous solution (10 mL×2), 25% brine (10 mL). The organic layer was dried over MgSO₄, filtered, and concentrated. The crude product was purified by a silica gel, eluting with 100% heptanes to 70% heptane-30% EtOAc. The product's fractions were pooled together, and concentrated to ˜6 mL volume. The resulting slurry was mixed at RT for 2 h, and cooled to 0+/−5° C. for 1 h. The product was collected by filtration, and rinsed with 1 mL of ice-cold heptane. The product was dried under vacuum at 50° C. overnight. to yield (S)-5-((2R,5R)-5-(benzyloxymethyl)-1,4-dioxan-2-yl)-3-ethynyl-4,5-dihydroisoxazole (250 mg, 65% isolated yield). Enantimeric Excess (ee)=99.8% by Chiral HPLC; mp: 102-103° C., crystalline needle; MS-ESI: 302 (M+1), 319 (M+18) ¹HNMR in CDCl₃: δ3.10 (1H, d, J=5.2 Hz), 3.12 (1H, d, J=2.9 Hz), 3.37 (1H, s), 3.39-3.50 (4H, m), 3.54 (1H, m), 3.72 (1H, m), 3.85 (1H, dd, J=11.5, 2.6 Hz), 4.02 (1H, dd, J=11.5, 2.6 Hz), 4.51 (1H, m), 4.53 (2H, s), 7.30 (5H, m).

### Step 12: (4R,7R)-7-((S)-3-ethynyl-4,5-dihydroisoxazol-5-yl)-11,11-dimethyl-1,10,10-triphenyl-2,6,9-trioxa-10-siladodecan-4-ol (3b)

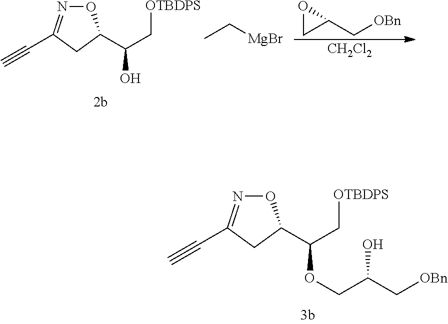

A 100 mL flask was charged with (R)-2-(tert-butyldiphenylsilyloxy)-1-((S)-3-ethynyl-4,5-dihydroisoxazol-5-yl)ethanol (4.40 g, potency=90.6%, 10.1 mmol, 1.0 equiv.), and CH₂Cl₂ (20 mL). The solution was stirred at 20° C. for 5 min, and cooled to 0° C. 3.38 mL (10.1 mmol, 1.0 equiv.) of 3.0 M ethylmagnesium bromide solution in diethyl ether was added slowly at the internal temperature of NMT 5° C. The resulting solution was stirred at 0° C. for 15 min, and the temperature allowed to rise to 20° C. over 30 min 4.15 g of benzyl-S-glycidyl ether (H₂O content by KF=360 ppm, 25.3 mmol, 2.5 equiv.) was added slowly (slightly exothermic). The reaction mixture was stirred at 20° C. for 5 min. The reaction mixture was heated to reflux, and mixed overnight. The reaction mixture were cooled to 20° C., and concentrated to ˜10 mL volume. The crude product was diluted with EtOAc (50 mL), washed with 10% citric acid aqueous solution (50 mL), and 25% brine (50 mL) respectively. The organic layer was dried over MgSO₄, filtered, and rinsed with EtOAc (5 mL). The filtrate was concentrated to ˜near dryness. Toluene (10 mL) was added, and the resulting crude product solution purified with silica gel column eluting with heptane to 75% heptane-25% EtOAc. The product's fractions were pooled together, and concentrated to dryness. The oil product was chased with 25 mL of EtOAc to dryness to give (4R,7R)-7-((S)-3-ethynyl-4,5-dihydroisoxazol-5-yl)-11,11-dimethyl-1,10,10-triphenyl-2,6,9-trioxa-10-siladodecan-4-ol as a light brown oil (3.84 g of the product, potency=73.5%; 50% isolated yield) MS-ESI: 558 (M+1); ¹HNMR in CDCl3: δ1.11 (9H, s), 2.53 (1H, d, J=4.7 Hz), 2.88 (1H, dd, J=16.8, 11.4 Hz), 3.11 (1H, dd, J=16.8, 8.1 Hz), 3.37 (1H, s), 3.4-3.9 (7H, m), 4.5-4.6 (3H, m), 4.86 (1H, ddd, J=11.2, 8.1, 3.0 Hz) 7.3-7.7 (15H, m).

### Step 13: (R)-1-(benzyloxy)-3-((R)-1-((S)-3-ethynyl-4,5-dihydroisoxazol-5-yl)-2-hydroxyethoxy)propan-2-ol (4b)

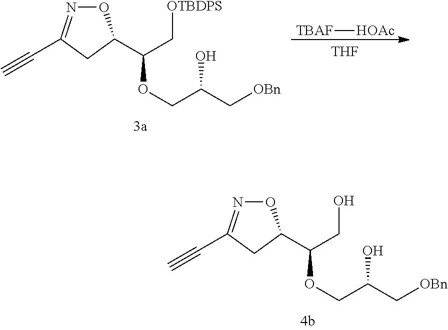

A 250 mL round bottom flask was charged with (4R,7R)-7-((S)-3-ethynyl-4,5-dihydroisoxazol-5-yl)-11,11-dimethyl-1,10,10-triphenyl-2,6,9-trioxa-10-siladodecan-4-ol (3b) 4.50 g, potency=74.0, 6.0 mmol, 1.0 equiv.), THF (35 mL), and acetic acid (0.537 g, 9.0 mmol, 1.5 equiv.). The mixture was stirred at 20° C. for 15 min or until all of the oil dissolved. The solution was cooled to −5° C. 9.0 mL (6 0 mmol) of 1M TBAF solution in THF was added slowly at the internal temperature of <10° C. The solution was stirred at 0° C. for 30 min and adjusted to 20° C. over 30 min. The solution was stirred at 20° C. for NLT 1 h. The solution was concentrated to ˜15 mL volume, and loaded to a silica gel column, eluted with heptane to 55% heptane-45% acetone. The product's fractions (#17-30) were pooled together and concentrated to dryness, chased with CH₂Cl₂ to dryness to give (R)-1-(benzyloxy)-3-((R)-1-((S)-3-ethynyl-4,5-dihydroisoxazol-5-yl)-2-hydroxyethoxy)propan-2-ol as an oil, 2.00 g (potency=90.7%, purity=98%). MS-ESI: 320 (M+1); ¹HNMR in CDCl3: δ2.85 (2H, s, br), 3.02 (1H, dd, J=17.2, 11.1 Hz), 3.11 (1H, dd, J=17.2, 8.2 Hz), 3.36 (1H, s), 3.42 (1H, dd, J=9.6, 6.2 Hz), 3.50 (1H, dd, J=9.6, 4.3 Hz), 3.52-3.65 (3H, m), 3.74 (2H, td, J=10.6, 3.4 Hz), 3.98 (1H, m), 4.53 (2H, s), 4.71 (1H, ddd, J=10.9, 8.1, 4.3 Hz), 7.31 (5H, m).

### Step 14: (R)-2-((R)-3-(benzyloxy)-2-hydroxypropoxy)-2-((S)-3-ethynyl-4,5-dihydroisoxazol-5-yl)ethyl 4-methylbenzenesulfonate (5b)

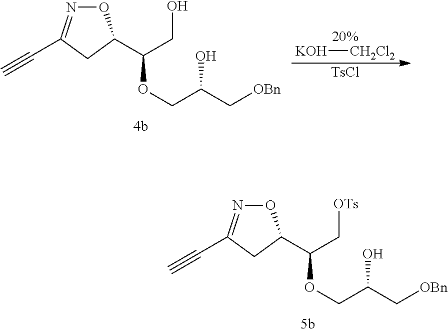

A 250 mL three-neck flask was charged with (R)-1-(benzyloxy)-3-((R)-1-((S)-3-ethynyl-4,5-dihydroisoxazol-5-yl)-2-hydroxyethoxy)propan-2-ol (1.95 g, potency=90.7%, 5.5 mmol, 1.0 equiv.), DCM (30 mL). The mixture was stirred at 20° C. for ˜15 min or until all of the oil dissolved. 20 wt % of potassium hydroxide solution (23.0 g, 82.0 mmol, 15.0 equiv.) was added, followed by slow addition of a solution of tosyl chloride (1.09 g, 5.5 mmol, 1.0 equiv.) in CH₂Cl₂ (5.0 mL) over 2 h at 20° C. The reaction mixture was stirred at 20° C. for NLT 1 h. The mixture was allowed to settle for 15 min, and the lower organic phase separated. The organic phase was washed with 12% brine (35 mL), dried over MgSO₄, filtered, and rinsed with CH₂Cl₂ (5 mL). The filtrate was concentrated to dryness. Toluene (5.0 mL) was added, and the crude product solution purified with a silica gel column, eluting with 100% heptane to 60% heptane-40% acetone. The product fractions were pooled together, concentrated to dryness. The product was chased with toluene (20 mL x 2) to dryness to remove any residual acetone to give (R)-2-((R)-3-(benzyloxy)-2-hydroxypropoxy)-2-((S)-3-ethynyl-4,5-dihydroisoxazol-5-yl)ethyl 4-methylbenzenesulfonate (2.53 g, potency=87.3wt %, potency adjusted yield=85%): MS-ESI: 474 (M+1); ¹HNMR in CDCl₃ δ2.25 (1H, t, J=6.6 Hz, OH), 2.44 (3H, s), 3.04 (2H, m), 3.36 (1H, s), 3.48 (2H, m),. 3.5-3.7 (3H, m), 3.87 (1H, m), 4.04 (1H, dd, J=10.8, 4.8 Hz), 4.14 (1H, dd, J=10.8, 4.1 Hz), 4.53 (2H, s), 4.69 (1H, ddd, J=10.8 , 7.8, 4.8 Hz), 7.1-7.4 (10H, m).

### Step 15: (S)-5-((2R,5R)-5-(benzyloxymethyl)-1,4-dioxan-2-yl)-3-ethynyl-4,5-dihydroisoxazole

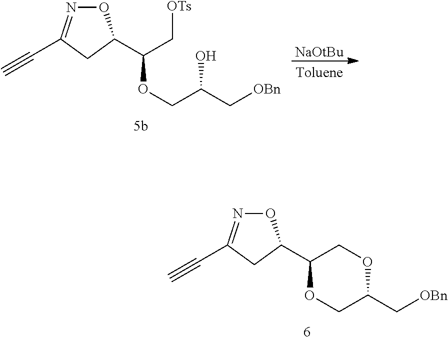 [SMILES: C#CC1=NO[C@H]([C@@H](COS(=O)(=O)c2ccc(C)cc2)OC[C@H](O)COCc2ccccc2)C1 ; C#CC1=NO[C@H]([C@H]2CO[C@H](COCc3ccccc3)CO2)C1 ; "NaOtBu"]

A 250 mL three-neck flask was charged with (R)-2-((R)-3-(benzyloxy)-2-hydroxypropoxy)-2-((S)-3-ethynyl-4,5-dihydroisoxazol-5-yl)ethyl-4-methylbenzene sulfonate (2.50 gm, potency=87.3%, 4.6 mmol, 1.00 equiv.), and toluene (45 mL, KF=75 ppm). The mixture was stirred at 20° C. for ˜15 min or until all of the oil dissolved. The solution was cooled to −10° C., and sodium tert-butoxide (0.488 g, 5.0 mmol, 1.08 equiv.) was added in one portion. The resulting slurry was mixed at 0° C. for NLT 3 h or until the starting material was less than 3% by HPLC. The mixture was quenched by addition of 10% brine solution (40 mL) at <10° C. The reaction mixture was warmed to 20° C., and mixed for 15 min. The upper organic phase was separated. The organic phase was washed with 5% NaHCO₃ aqueous solution (25 mL), and 25% brine (25 mL) respectively. The organic layer was dried over MgSO₄, filtered, and rinsed with toluene (5 mL). The filtrate was concentrated to ˜7 mL volume, and the solution purified with a 40 g silica gel column, eluting with 100% heptane to 70% heptane-30% EtOAc. The product's fractions were poole together, and concentrated to ˜20 mL volume. The resulting slurry was mixed at 20° C. for 1 h. The slurry was cooled to 0° C., and stirred for 1 h more. The product was collected by filtration, and rinsed with ice-cold heptane (5 mL), dried at 40° C. under vacuum overnight to give (S)-5-((2R,5R)-5-(benzyloxymethyl)-1,4-dioxan-2-yl)-3-ethynyl-4,5-dihydroisoxazole as a white and crystalline solid (1.08 g, 78% isolated yield): mp: 102-103° C.; MS-ESI: 302 (M+1), 319 (M+18); ¹HNMR in CDCl₃: 3.10 (1H, d, J=5.2 Hz), 3.12 (1H, d, J=2.9 Hz), 3.37 (1H, s), 3.39-3.50 (4H, m), 3.54 (1H, m), 3.72 (1H, m), 3.85 (1H, dd, J=11.5, 2.6 Hz), 4.02 (1H, dd, J=11.5, 2.6 Hz), 4.51 (1H, m), 4.53 (2H, s), 7.30 (5H, m).

1. A compound of Structural Formula (I): 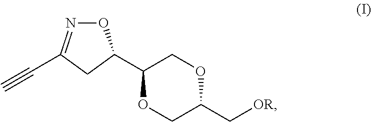
wherein R is —H or a hydroxyl protecting group.

2. The compound of claim 1, wherein R is H, optionally substituted methyl, optionally substituted ethyl, or optionally substituted benzyl.

3. (canceled)

4. A method of preparing a compound of claim 2,
the method comprising: cyclizing a compound of Structural Formula (IIa) or (IIb): 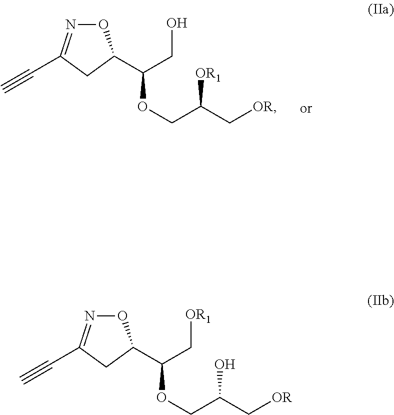
in the presence of a base, wherein:
R₁ is a hydroxyl activation group.

5. (canceled)

6. The method of claim 4, wherein R₁ is C₁₋₁₀ alkylsulfonate, or C₁₋₁₀ arylsulfonate.

7. The method of claim 4, wherein R₁ is mesylate, triflate, nonaflate, tresylate, besylate, nosylate, brosylate, or tosylate.

8. (canceled)

9. The method of claim 6, wherein the base is an alkali metal hydroxide, an alkali metal C₁₋₆alkoxide, or an alkali metal bis(trimethylsilyl)amide.

10. (canceled)

11. The method of claim 9, wherein R is benzyl and R₁ is tosylate.

12. A compound of Structural Formula (III): 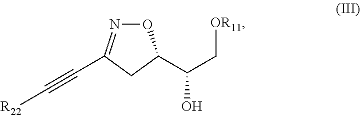
wherein:
R₁₁ is —H or a hydroxyl protecting group; and
R₂₂ is H, trimethylsilyl (TMS), triethylsilyl (TES), triisopropylsilyl (TIPS), dimethylisopropylsilyl (DMIPS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), biphenyldimethylsilyl, triisopropylsilyl, biphenyldiisoporpylsilyl, or 2-(2-hydroxypropyl).

13. The compound of claim 12, wherein R₁₁ is t-butyldimethylsilyl (TBDMS), t-butyldiphenylsilyl (TBDPS), or trityl; and R₂₂ is trimethylsilyl (TMS), triethylsilyl (TES), t-butyldimethylsilyl (TBDMS), dimethylthexylsilyl, biphenyldimethylsilyl, triisopropylsilyl, biphenyldiisoporpylsilyl, or 2-(2-hydroxypropyl).

14-15. (canceled)

16. A method of preparing a compound of Structural Formula (III): 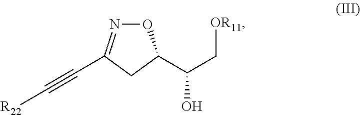
the method comprising: reacting a compound of Structural Formula (IV): 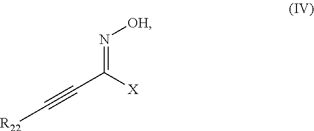
and a compound of Structural Formula (V) in the presence of a C₁₋₆ alkyl magnesium halide and a C₁₋₆alcohol, 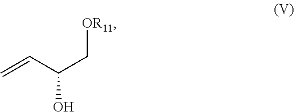
wherein:
X is halide;
R₁₁ is —H or a hydroxyl protecting group; and
R₂₂ is H, trimethylsilyl (TMS), triethylsilyl (TES), triisopropylsilyl (TIPS), dimethylisopropylsilyl (DMIPS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), biphenyldimethylsilyl, triisopropylsilyl, biphenyldiisoporpylsilyl, or 2-(2-hydroxypropyl).

17-18. (canceled)

19. The method of claim 16, wherein R₁₁ is t-butyldimethylsilyl (TBDMS), t-butyldiphenylsilyl (TBDPS), or trityl; and R₂₂ is trimethylsilyl (TMS), triethylsilyl (TES), t-butyldimethylsilyl (TBDMS), dimethylthexylsilyl, biphenyldimethylsilyl, triisopropylsilyl, biphenyldiisoporpylsilyl, or 2-(2-hydroxypropyl).

20-21. (canceled)

22. The method of claim 16, further comprising a step of removing R₂₂ of the compound of Structural Formula (III), thereby forming a compound of Structural Formula (VI):

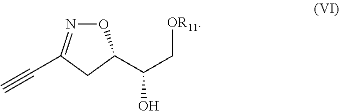

23. (canceled)

24. The method of claim 22, further comprising: reacting a compound of Structural Formula (VI) with a carboxylic acid R₃₃COOH to form an ester of Structural Formula (VII) via Mitsunobu inversion: 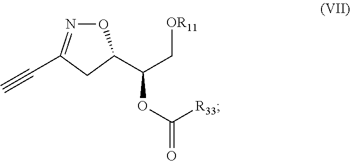
and converting the ester into an alcohol of Structural Formula (VIII):

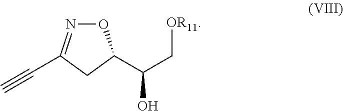

25. The method of claim 24, wherein the reacting step is conducted in the presence of an azodicarboxylate and triphenylphosphine (TPP).

26-27. (canceled)

28. The method of claim 24, further comprising a step of reacting the compound of Structural Formula (VIII) with an epoxide 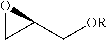
to form a compound of Structural Formula (IXa): 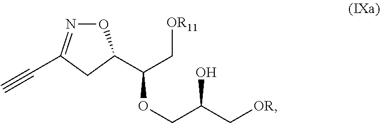
or reacting the compound of Structural Formula (VIII) with an epoxide 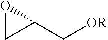
to form a compound of Structural Formula (IXb): 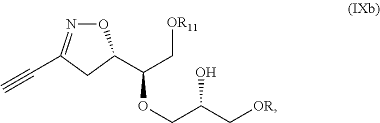
wherein R is —H or a hydroxyl protecting group.

29. The method of claim 28, wherein the reacting step is conducted in the presence of C₁₋₆ alkyl magnesium halide.

30. (canceled)

31. The method of claim 28, wherein R is H, optionally substituted methyl, optionally substituted ethyl, or optionally substituted benzyl.

32. (canceled)

33. The method of claim 28, further comprising a step of converting the compound of Structural Formula (IXa) into a compound of Structural Formula (Xa) in the presence of an amine base: 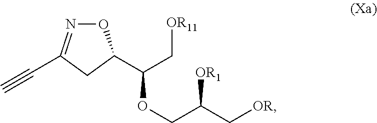
wherein R₁ is a hydroxyl activating group.

34. (canceled)

35. The method of claim 33, wherein R₁ is C₁₋₁₀ alkylsulfonate or C₁₋₁₀ arylsulfonate.

36. (canceled)

37. The method of claim 33, further comprising a step of converting the compound of Structural Formula (Xa) into a compound of Structural Formula (IIa) in the presence of an acid:

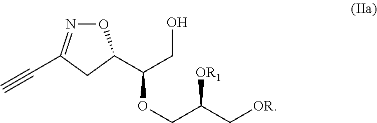

38-42. (canceled)

43. The method of claim 37, wherein R₁₁ is t-butyldiphenylsilyl (TBDPS).

44-48. (canceled)

49. The method of claim 28, further comprising a step of converting the compound of Structural Formula (IXb) into a compound of Structural Formula (Xb) in the presence of an acid:

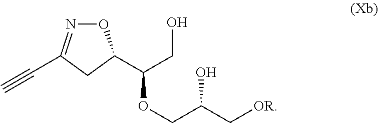

50-53. (canceled)

54. The method of claim 49, wherein R₁₁ is t-butyldiphenylsilyl (TBDPS) and R is benzyl.

55. The method of claim 49, further comprising a step of converting the compound of Structural Formula (Xb) into a compound of Structural Formula (IIb): 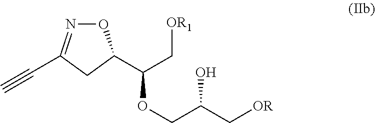
wherein R₁ is a hydroxyl activating group.

56. The method of claim 55, wherein R₁ is C₁₋₁₀ alkylsulfonate or C₁₋₁₀ arylsulfonate.

57-63. (canceled)
